(19) United States
(12) Patent Application Publication
(10) Pub. No.: US 2025/0263585 A1
(43) Pub. Date: Aug. 21, 2025

(54) ADHESIVE PRODUCT MADE OUT OF PYROLYSIS OIL VIA A POLYMERIZATION

(71) Applicant: TOTALENERGIES ONETECH, Courbevoie (FR)

(72) Inventors: Benjamin BIBER, Exton, PA (US); Christine RICHARDSON, Paris La Défense Cedex (FR); Keith NELSON, Aurora, CO (US)

(21) Appl. No.: 18/994,027

(22) PCT Filed: Jul. 13, 2023
(86) PCT No.: PCT/EP2023/069567

§ 371 Date: Jan. 13, 2025

(30) Foreign Application Priority Data
Jul. 14, 2022 (EP) 22315146.5
Jul. 14, 2022 (EP) 22315147.3
Jul. 14, 2022 (EP) 22315148.1
Jul. 14, 2022 (EP) 22315149.9
Jul. 14, 2022 (EP) 22315150.7
Jul. 14, 2022 (EP) 22315151.5
Jul. 14, 2022 (EP) 22315152.3
Jul. 14, 2022 (EP) 22315153.1

(51) Int. Cl. C09J 4/06; C10G 7/00; C10G 50/00

(52) U.S. Cl. CPC C09J 4/06; C10G 7/006; C10G 50/00

(57) ABSTRACT

The invention relates to an adhesive composition comprising an elastomer and tackifying resins wherein one of said tackifying resins comprises a liquid resin prepared from a liquified waste polymer, being preferably a pyrolysis oil via polymerization preferably prepared via cationic polymerization.

### FIELD OF THE DISCLOSURE

The disclosure relates to an adhesive composition comprising a liquid resin derived from the polymerization of pyrolysis oil. In particular, the disclosure relates to the polymerization of the polymerizable components in the pyrolysis oil preferably via polymerization in order to obtain a low molecular weight oligomeric product that can be used as a liquid tackifier that can function as a processing oil in adhesive compositions.

### BACKGROUND OF THE DISCLOSURE

Due to its increased availability, low-cost gas is displacing liquids as the feedstock in the steam cracking process to produce ethylene. The result is that lower amounts of C₅ diolefins are being produced leading to a shortage of this monomer to produce tackifying resins. There is therefore a need for alternative monomers for the augmentation or even complete displacement of current petroleum-based diolefins to produce tackifying resins that are cost-effective and readily available.

Waste plastics are mostly diverted to landfills or are incinerated, with a smaller fraction being diverted to recycling. There is however a strong need, influenced by the regulations to limit waste plastic in landfills. On the other hand, waste plastics disposal into landfills is becoming increasingly difficult. There is therefore a need for recycling waste plastics.

A possible route to recycle plastic is via plastic pyrolysis. However, the pyrolysis plastic oil obtained from the plastic pyrolysis generally is generally not easily valorised. The pyrolysis plastic oil contains large quantities of impurities and also large quantities of dienes and of olefins.

U.S. Pat. No. 3,809,680 describes a hard resin prepared by the reaction of formaldehyde and pyrolysis oil obtained by the destructive distillation of scrap rubber vulcanizate and its preparation. The resins are useful as a tackifier to be used in elastomers.

U.S. Pat. No. 3,933,720 relates to polychloroprene type adhesive compositions comprising an acid-modified hydrocarbon resin, polychloroprene, and suitable adhesive such as metal oxide and anti-aging agents together with, if desired, known resins for polychloroprene type adhesives, such as alkylphenol resins and petroleum resins, and a volatile solvent.

US2012/0214592 relates to an adhesive composition made from an elastomer and a tackifying resin. The tackifying resin includes a farnesene polymer or copolymer having the following properties: i) less than 10 weight percent of volatile organic compounds; ii) Mn between 300 Da and 1000 Da; iii) Mw between 400 Da and 3000 Da; iv) Mw/Mn between 1.00 and 3.00; v) Tg between −50° C. and 20° C.; and vi) viscosity between 400,000 cP and 1,000,000 cP at 25° C. A method of making the farnesene-based polymer or copolymer includes combining a farnesene monomer and a solvent and optionally adding one or more co-monomers selected from dienes, branched mono-olefins, and vinyl aromatics, to provide a monomer feed, and polymerizing the monomer feed by combining it with a Friedel-Crafts initiator in a vessel. The farnesene-based polymer or copolymer tackifier may be combined with one or more elastomers and one or more other tackifiers to form an adhesive composition.

FR3,116,279 relates to a method for producing hydrocarbon resins from a feedstock containing rubber chips, the method comprising at least one pyrolysis step and a resin synthesis step.

There is therefore a need for the valorization of those pyrolysis plastic oil. More precisely, there is clearly a need for the valorization of the olefins and of the dienes present in the pyrolysis plastic oils.

### SUMMARY OF THE DISCLOSURE

The aim of the present disclosure is to provide a valorization route for the olefins and dienes that are present in the pyrolysis oil. More precisely, the aim of the present disclosure is to convert the polymerizable materials present in the pyrolysis oil into valuable oligomeric products. In particular, it was found that it is possible to produce adhesive composition from liquid resin prepared from a liquified waste polymer.

It has been discovered that the oligomeric products prepared from pyrolysis oil, and especially from pyrolysis plastic oil, are particularly advantageous. Indeed, such oligomeric products present a relatively low viscosity. With low viscosity, it is easier to handle the oligomeric product. In particular, it is easier to mix the liquid resin or oligomeric product with other components. In particular, less mechanical energy is necessary to mix the liquid resin or oligomeric product with other components. Another advantage of the liquid resin or oligomeric product lies in its content in aromatics. Indeed, having some aromatics allows to improve the compatibility of the liquid resin or oligomeric product with certain other constituents in an adhesive composition.

According to a first aspect, an adhesive composition comprising an elastomer and one or more tackifying resins is remarkable in that one of the one or more tackifying resins comprises a liquid resin prepared from a liquified waste polymer, being preferably a pyrolysis oil via polymerization.

As it will be clear in the following, the liquid resin is an oligomeric product.

In a preferred embodiment, the adhesive composition elastomer is selected from the group consisting of styrene-isoprene block copolymers, polyacrylate resins, polyethylene vinyl acetate (EVA) resins, polystyrene butadiene resins, random styrenebutadiene (SBR) copolymers, styrene-butadiene block copolymers, styrene-isoprene-butadiene-styrene (SIBS) copolymers, styrene-ethylene-propylene-styrene (SEPS) copolymers, styrene-ethylene-butylene-styrene (SEBS) block copolymers, amorphous poly-olefin (APO) resins, and mixtures thereof.

In some embodiments, the adhesive composition content of said elastomer ranges from 1 to 90 wt. %.

In some embodiments, the content of tackifying resin derived from liquid waste polymer ranges from 1 to 75 wt. %; preferably from 5 to 60 wt. %.

In some embodiments, the liquid resin (i.e., the oligomeric product) shows an olefinic content of at most 20.0 wt. % as determined by 1H NMR. With preference, the liquid resin an olefinic content ranging from 0.1 to 20.0 wt. %; preferably from 0.3 to 15.0 wt. %; more preferably, from 0.5 to 10.0 wt. %. from 0.8 to 5.0 wt. %.

In some embodiments, the liquid resin (i.e., the oligomeric product) shows an aliphatic content ranging from 15 to 100 wt. % as determined by 1H NMR; preferably ranging from 40.0 to 99.5 wt. %; more preferably ranging from 50.0 to 99.0 wt. %; even more preferably ranging from 70.0 to 98.5 wt. %; and most preferably ranging from 80.0 to 98.0 wt. %.

In one or more embodiments, the liquid resin (i.e., the oligomeric product) shows an aromatic content ranging from 0.3 to 45.0 wt. % as determined by 1H NMR; preferably ranging from 0.5 to 40.0 wt. %; more preferably ranging from 0.8 to 30.0 wt. %; and even more preferably ranging from 1.0 to 20.0 wt. %.

In embodiments, the liquid resin (i.e., the oligomeric product) has a viscosity at 30° C. ranging from 10 to 50 000 cps measured using a Brookfield viscometer; preferably ranging from 10 to 50 000 cps; more ranging from 10 to 10 000 cps; even more preferably ranging from 10 to 4 000 cps; and most preferably ranging from 10 to 2 500 cps.

In some embodiments, the liquid resin (i.e., the oligomeric product) has a number average molecular weight (Mn) ranging from 100 to 10,000 g/mol measured using gel permeation chromatography and a polystyrene calibration; preferably from 120 to 5,000 g/mol; more preferably from 140 to 1,000 g/mol; even more preferably from 150 to 800 g/mol; most preferably from 180 to 700 g/mol; or from 200 to 650 g/mol; or from 250 to 550 g/mol.

In some embodiments, the liquid resin (i.e., the oligomeric product) has a weight average molecular weight (Mw) ranging from 100 to 10,000 g/mol measured using gel permeation chromatography and a polystyrene calibration; preferably from 120 to 5,000 g/mol; more preferably from 150 to 3,500 g/mol; and even more preferably from 200 to 2,000 g/mol.

In some embodiments, the liquid resin (i.e., the oligomeric product) has a z-average molecular weight (Mz) ranging from 100 to 100,000 g/mol measured using gel permeation chromatography and a polystyrene calibration; preferably from 120 to 85,000 g/mol; more preferably from 150 to 70,000 g/mol; and even more preferably from 200 to 50,000 g/mol.

In some embodiments, the liquid resin (i.e., the oligomeric product) has a Mw/Mn ranging from 1.0 to 10.0; preferably from 1.0 to 5.0; more preferably from 1.0 to 4.0; and even more preferably from 1.1 to 2.8.

In a preferred embodiment, the liquid resin (i.e., the oligomeric product) comprises a comonomer selected from the group consisting of styrene, alpha-methyl styrene, 2-methyl styrene, 3-methyl styrene, 4-methyl styrene, 4-t-butyl styrene, 4-cyclohexyl styrene, 4-dodecyl styrene, 2,4-diisopropyl styrene, 2.4.6-trimethyl styrene, 2-ethyl-4-benzylstyrene, 4-(phenylbutyl) styrene, 1-vinyl naphthalene, 2-vinyl naphthalene, vinyl anthracene, 4-methoxy styrene, monochlorostyrene, dichlorostyrene, divinyl benzene, Indene, methyl-Indene, and mixtures thereof; with preference the liquid resin (i.e., the oligomeric product) comprises a comonomer being or comprising styrene.

In embodiments, the liquid resin (i.e., the oligomeric product) comprises a comonomer present at a concentration ranging from 0.01 to 90 mol. % based on the total weight of the liquid resin, preferably from 1.0 to 80 mol. %.

In some embodiments, the liquid resin (i.e., the oligomeric product) is a wax and/or shows a crystallisation temperature (Tc) below 35° C.; preferably ranging from 18 to 32° C.; more preferably ranging from 20 to 30° C.; and even more preferably ranging from 21 to 29° C.

In embodiments, the liquid resin (i.e., the oligomeric product) is a resin and/or shows a glass transition temperature (Tg) below −20° C. as determined by Differential Scanning calorimetry; preferably ranging from −60° C. to −25° C.; more preferably from −50° C. to −30° C.; and even more preferably from −49° C. to −35° C.

According to a second aspect, the disclosure relates to an article comprising a substrate and the adhesive composition according to the first aspect; the adhesive composition being applied to at least one surface of the substrate, wherein the substrate is selected from the group consisting of a tape and a label.

According to a third aspect, the disclosure relates to a coated article comprising the adhesive composition according to the first aspect; the adhesive composition being applied to at least one of wood, a non-woven fabric, and a book-binding.

According to a fourth aspect, the disclosure relates to a case and carton assembly line comprising an adhesive station, wherein the adhesive station dispenses an adhesive composition according to the first aspect.

According to a fifth aspect, the disclosure relates to a process for the preparation of a liquid resin from a liquified waste polymer, the liquid resin being an oligomeric product and being suitable to prepare an adhesive composition according to the first aspect or in any product according to any of the second to fourth aspect, the process is remarkable in the following steps:
a) providing a feedstream containing liquified waste polymer, wherein said feedstream contains at least 0.1 wt. % to at most 80 wt. % of dienes based on the total weight of said feedstream;
c) performing a polymerization reaction on said feedstream under polymerization conditions to obtain a first product stream comprising an oligomeric product and a purified liquified waste polymer;
d) optionally, performing a neutralization reaction by contacting said first product stream with a basic compound to obtain a neutralized product stream and removing said basic compound from neutralized product stream to obtain a second product stream;
g) performing a separation to separate the oligomeric product from the purified liquified waste polymer.

In some embodiments the liquified waste polymer is a pyrolysis plastic oil, with preference, step a) of providing a feedstream containing liquified waste polymer comprises the preliminary steps of preparation of liquified waste polymer including:
a1) of providing a waste plastics stream;
a2) pyrolyzing said waste plastics stream at a temperature of at least 200° C.;
a3) recovering a pyrolizer effluent and separating, into a C₁ to C₄ hydrocarbons fraction, a fraction having a boiling range higher than 350° C. and a remaining fraction being said pyrolysis plastic oil;
a4) optionally sending the fraction having a boiling range higher than 350° C. into a Fluid Catalytic Cracking (FCC) unit, or a hydrocracking unit, a coker or a visbreaker or blending said fraction having a boiling range higher than 350° C. in crude oil or in a crude oil cut to be further refined.

In one or more embodiments, the process further comprises a step b) of drying the feedstream to obtain a dried feedstream wherein step b) is performed before step c) of polymerization so that step c) of polymerization reaction is performed on the dried feedstream. With preference, step b) of drying is performed and comprises a sub-step b1) of decantation and/or centrifugation. More preferably, the first sub-step b1) is followed by a second sub-step b2) of drying using a molecular sieve to reach a water content of less than 0.1 vol. % according to ASTM D95-13 (2018).

In some embodiments, the feedstream contains at least 75 wt. % of the liquefied waste polymer based on the total weight of the feed stream.

Step c) comprises performing a polymerization reaction preferably a cationic polymerization or a free radical polymerization or an anionic polymerization.

In a preferred embodiment, the polymerization reaction is a cationic polymerization; more preferably, the polymerization reaction in step c) is a cationic polymerization performed in the presence of an acidic catalyst; with preference, the acidic catalyst is a Brönsted acid or a Lewis acid.

In a preferred embodiment, the polymerization reaction in step c) is a cationic polymerization performed in the presence of an acidic catalyst being a Lewis acid chosen among BF₃, complexes of boron trifluoride, AlCl₃, SnCl₄, ZnCl₂, FeCl₃ and TiCl₃, alkyl aluminum chlorides, H₂SO₄ or any mixture thereof; with preference, acidic catalyst is or comprises boron trifluoride etherate.

In embodiments, the acidic catalyst is present at a concentration ranging from 0.5 wt. % to 5.0 wt. % based on the total weight of said feedstream and/or the polymerization reaction of step c) is carried out until the dienes of the purified liquified waste polymer is less than 5.0 wt. % based on the total weight of the first product stream.

In a preferred embodiment, the polymerization conditions of step (c) comprise a contact time ranging from 5 min to 5 hours; and/or a temperature ranging from 5 to 100° C. at atmospheric pressure

In embodiments, the polymerization reaction on step c) is performed in the presence of one or more comonomers; with preference, the one or more comonomers comprise a vinyl aromatic and/or the one or more comonomers are present at a concentration from 1.0 to 25.0 wt. % based on the total weight of the liquified waste polymer.

In some embodiments, the basic compound forms a basic stream; wherein the basic stream and the first product stream are contacted with a weight ratio ranging from 1:1 to 1:1,000.

In some embodiments, step d) is performed and the concentration of the basic compound ranges from 0.1 to 50.0 wt. % based on the total weight of said neutralized product stream.

In a preferred embodiment, step d) is performed and the basic compound:
has a pKa in water ranging from 7.5 to 14; and/or
is selected from LiOH, NaOH, CsOH, Ba(OH)₂, Na₂O, KOH, K₂O, CaO, Ca(OH)₂, MgO, Mg(OH)₂, NH₄OH or any mixtures thereof.

In embodiments, step d) is performed in continuous mode and/or the removal of said basic compound from neutralized product stream, to obtain a second product stream, is performed by decantation and/or by centrifugation.

In embodiments, the process further comprises a step e) of washing the first product stream or the second product stream with a solvent to obtain a washed stream; with preference, the washing is performed at a temperature ranging from 5° C. to 95° C.

In a preferred embodiment, the solvent is selected from water or an aqueous acidic solution comprising:
one or more organic acids selected from citric acid (C₆H₈O₇), formic acid (CH₂O₂), acetic acid (CH₃COOH), sulfamic acid (H₃NSO₃) and any combination thereof; and/or
one or more inorganic acids selected from hydrochloric acid (HCl), nitric acid (HNO₃), sulfuric acid (H₂SO₄), phosphoric acid (H₃PO₄), and any combination thereof.

In some embodiments, the washing is performed until the pH of said washed stream is in the range of 5.0 to 9.0; and/or the washing is followed by decantation and/or centrifugation to separate the solvent from the washed stream.

In embodiments, the process further comprises a step f) of filtering the stream obtained in the previous step to obtain a filtered stream wherein the filtering is performed to remove solids from the first product stream or from the second product stream or from the washed stream, and/or to coalesce remaining traces of solvent if any; with preference, the filtering step is followed by a dewatering step.

In embodiments, step g) of separation is performed via distillation or steam distillation or vacuum stripping or fractional distillation or any combination.

In a preferred embodiment, the process after the separation step g), the purified liquified waste polymer is recovered and blended in the fuel pool; with preference, the purified liquified waste polymer is separated in a naphtha cut having a boiling range of less than 150° C. and a diesel cut having a boiling range between 15° and 350° C., wherein said naphtha cut is incorporated in a naphtha pool, said diesel cut is incorporated in a diesel pool.

In a preferred embodiment, the liquified waste polymer in the feedstream has a final boiling point of at most 700° C.

In a preferred embodiment, the feedstream contains from 0.1 to 50.0 wt. % of dienes based on the total weight of said feedstream; preferably from 0.1 to 25.0 wt. %.

According to a sixth aspect, the disclosure provides an installation for carrying out a process of the fifth aspect, said installation is remarkable in that it comprises:
a polymerization section;
an optional neutralization section;
a separation section;
wherein the polymerization section and the separation section are fluidically connected in series, and wherein the neutralization section when present is placed downstream the polymerization section and upstream the separation section.

With preference, the installation comprises:
a pre-treatment section comprising a pyrolysis unit, one or more separation units and an optional washing unit;
a drying section comprising a decanter and/or a centrifuge; and/or a molecular sieve;
a polymerization section comprising one or more polymerization reactors; preferably loaded with an acidic catalyst;
a neutralization section a mixing reactor and an optional separation unit comprising at least one selected from a decanter, a centrifuge, and a filter;
a washing section comprising a mixing vessel and a separation unit comprising a decanter and/or a centrifuge;
a filtering section comprising a filter and an optional molecular sieve.
a separation section comprising a separation unit comprising one or more distillation columns.

In a seventh aspect, the disclosure provides a method to produce an adhesive composition comprising the production of an oligomeric product according to the process of the fifth aspect; and mixing the recovered oligomeric product an elastomer to form an adhesive composition; wherein the oligomeric product is a liquid resin prepared from a liquified waste polymer and used as a tackifying resin.

### DESCRIPTION OF THE FIGURES

FIG. 1 describes a possible embodiment of the invention. In this possible embodiment, the liquified waste polymer is firstly optionally dried, and sent to the cationic polymerization to convert the dienes into oligomeric products. The effluents of the cationic polymerization are then sent to an optional treatment with a basic compound followed by the removal of the basic compound and followed by an optional washing step. After an optional filtering step, the various fractions are separated into various fraction depending on the boiling point. If a heavy fraction can be separated, it is sent to conversion units. The oligomeric product that is converted into resins can also be separated. If a remaining fraction of purified plastic oil fraction is present it can be sent to an hydrotreatment or a cracking or a steam cracking to produce olefins being further polymerized. The oligomeric product obtained can also be used as valuable resins.

FIG. 2 presents the calibration curve for maleic anhydride concentration.

FIG. 3 presents the 1H NMR olefinic content of treated pyrolysis oil compared to crude pyrolysis oil of examples 2 to 4.

FIG. 4 presents the olefinic carbon stretching peak areas from FTIR of examples 2 to 4.

FIG. 5 presents the viscosity of Adhesive Formulations at 160° C. relating to example 6

FIG. 6 presents the 180° Peel Adhesion Testing on SS relating to example 6.

FIG. 7 presents the 180° Peel Adhesion Testing on HDPE relating to example 6

FIG. 8 presents the 180° Peel Adhesion Testing on CC relating to example 6.

FIG. 9 presents the Loop Tack Results on SS relating to example 6.

FIG. 10 presents the Loop Tack Results on HDPE relating to example 6

FIG. 11 presents the Loop Tack Results on CC relating to example 6

FIG. 12 presents the Quick Stick Results on SS relating to example 6

FIG. 13 presents the Quick Stick Results on HDPE relating to example 6

FIG. 14 presents the Quick Stick Results on CC relating to example 6

FIG. 15 presents the Room Temperature Shear Results on SS relating to example 6

FIG. 16 presents the room Temperature Shear Results on Kraft Paper relating to example 6.

FIG. 17 presents the SAFT Results on SS relating to example 6

FIG. 18 presents the SAFT Results on Kraft Paper relating to example 6

FIG. 19 is an example of installation according to the disclosure.

### DEFINITIONS AND ANALYTICAL METHODS

For the purpose of the disclosure, the following definitions are given:

The terms “alkane” or “alkanes” as used herein describe acyclic branched or unbranched hydrocarbons having the general formula CnH2n+2, and therefore consisting entirely of hydrogen atoms and saturated carbon atoms; see e.g. IUPAC. Compendium of Chemical Terminology, 2nd ed. (1997). The term “alkanes” accordingly describes unbranched alkanes (“normal-paraffins” or “n-paraffins” or “n-alkanes” or “paraffins”) and branched alkanes (“iso-paraffins” or “iso-alkanes”) but excludes naphthenes (cycloalkanes). They are sometimes referred to by the symbol “HC—”.

The terms “olefin” or “alkene” as used herein relate to an unsaturated hydrocarbon compound containing at least one carbon-carbon double bond. They are sometimes referred to by the symbol “HC═”.

The term “hydrocarbon” refers to the alkanes (saturated hydrocarbons), cycloalkanes, aromatics and unsaturated hydrocarbons together.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The recitation of numerical ranges by endpoints includes all integer numbers and, where appropriate, fractions subsumed within that range (e.g. 1 to 5 can include 1, 2, 3, 4 when referring to, for example, a number of elements, and can also include 1.5, 2, 2.75 and 3.80, when referring to, for example, measurements). The recitation of endpoints also includes the recited endpoint values themselves (e.g. from 1.0 to 5.0 includes both 1.0 and 5.0). Any numerical range recited herein is intended to include all sub-ranges subsumed therein.

The term “conversion” means the mole fraction (i.e., percent) of a reactant converted to a product or products. The term “selectivity” refers to the percentage of converted reactants that went to a specified product.

The terms “wt. %”, “vol. %”, or “mol. %” refers to the weight, volume, or molar percentage of a component, respectively, based on the total weight, the total volume of material, or total moles, that includes the component. In a non-limiting example, 10 grams of a component in 100 grams of the material is 10 wt. % of the component.

The term “naphtha” refers to the general definition used in the oil and gas industry. In particular, it refers to a hydrocarbon originating from crude oil distillation having a boiling range from 15 to 250° C. as measured by ASTM D2887-22e1. Naphtha contains substantially no olefins as the hydrocarbons originate from crude oil. It is generally considered that a naphtha has a carbon number between C3 and C11, although the carbon number can reach in some cases C15. It is also generally admitted that the density of naphtha ranges from 0.65 to 0.77 g/mL.

The term “gasoil” refers to the general definition used in the oil and gas industry. In particular, it refers to a hydrocarbon having a boiling point in the range of 250 to 350° C. as measured according to the ASTM D7500-15(2019).

The term “liquified waste polymer” refers to any hydrocarbon liquid obtained from a waste polymer, preferably obtained from waste plastics. To obtain such liquified waste polymer, processes such as hydrothermal liquefaction or thermal pyrolysis or catalytic pyrolysis or steam pyrolysis or even pyrolysis in the presence of hydrogen of waste polymer can be used. The waste polymers encompass any polymer that is considered as waste such as waste polyethylene (PE), waste polypropylene (PP), waste polyethylene terephthalate (PET), waste polyvinyl chlorine (PVC) but also other polymers originating from waste or used tires for instance. The term “liquified waste polymer” also encompasses “pyrolysis plastic oil”.

The term “pyrolysis plastic oil” refers to the liquid products obtained once waste plastics have been thermally pyrolyzed. The pyrolysis process shall be understood as an unselective thermal cracking process. The pyrolysis involves the breaking of the polymer chains by heating. Rather than breaking the polymer down to its original monomers, pyrolysis tends to make a range of shorter-chain compounds, similar in many ways to the mixtures of hydrocarbons found in crude oil and oil products. A catalyst is sometimes used to reduce the operating temperature. The plastic being pyrolyzed can be of any type. For instance, the plastic being pyrolyzed can be polyethylene, polypropylene, polystyrene, polyesters, polyamides, polycarbonates, etc. These liquified waste polymers contain paraffins, i-paraffins (iso-paraffins), dienes, alkynes, olefins, naphthenes, and aromatic components. Liquified waste polymers may also contain impurities such as organic chlorides, organic silicon compounds, metals, salts, oxygenates, sulfur and nitrogen compounds, etc. The origin of the plastic is the waste plastic without limitation on the origin or on the nature of the plastic. The composition of the liquified waste polymer is dependent on the type of plastic pyrolyzed. It is however mainly constituted of hydrocarbons having from 1 to 50 carbon atoms and impurities.

The content in paraffin, naphthene, olefin, and aromatic is determined according to the standardized method UOP990-11. This method uses two-dimensional gas chromatography (GC×GC) coupled with a flame ionization detector (FID).

The term Diene Value (DV) or Maleic Anhydride Value (MAV) corresponds to the amount of maleic anhydride (expressed as equivalents of iodine) which will react with 100 parts of oil under specific conditions. It is a measure of the conjugated double bonds in the oil. One mole of Maleic anhydride corresponds to 1 conjugated double bond. One known method to quantify the diene is the UOP 32617: Diene Value by Maleic Anhydride Addition Reaction. The term diene value (DV) refers to the analytical method by titration expressed in g of iodine per 100 g of sample. The term Maleic Anhydride value (MAV) refers to the analytical method by titration expressed in mg of Maleic acid per g of sample. There is a correlation between the MAV=DV*3,863 since 2 moles of iodine correspond to 1 mole of Maleic Anhydride. For the examples presented, a particular method was used. The weight % of dienes presented in this application was obtained according to this particular method described in the example section.

The term bromine number corresponds to the amount of bromine in grams reacted by 100 grams of a sample. The number indicates the quantity of olefins in a sample. It is determined in grams of Br₂ per 100 grams of solution (gBr2/100 g) and can be measured for instance according to the method ASTM D1159-07(2017).

The term bromine index is the number of milligrams of bromine that react with 100 grams of sample. It is determined in milligrams of Br₂ per 100 g of solution (mg Br₂/100 g) and can be measured for instance according to the method ASTM D2710-20.

The term boiling point used refers to the boiling point generally used in the oil and gas industry. They are measured at atmospheric pressure. The initial boiling point is defined as the temperature value when the first bubble of vapor is formed. The final boiling point is the highest temperature that can be reached during a standard distillation. At this temperature, no more vapor can be driven over into the condensing units. The determination of the initial and the final boiling point is known per se in the art. Depending on the boiling range of the mixture they can be determine using various standardized methods such as for instance the ASTM D2887-22e1 relating to the boiling range distribution of petroleum fractions by gas chromatography. For compositions containing heavier hydrocarbons the ASTM D7169-20e1 can alternatively be used. The boiling ranges of the distillates can also advantageously be measure using the ASTM D7500-15 (2019) or the standard NF EN 15199-1 Due to the nature of the liquified waste polymer the preferred method for the measurement of the boiling point is the method NF EN 15199-1. Depending on the method, up to 10° C. of difference can be evidenced. Hence, the boiling point expressed here is to be considered with an absolute uncertainty of +/−10° C.

The molecular weight: Mn (number average molecular weight), Mw (weight average molecular weight), Mz (z average molecular weight) and molecular weight distribution d (Mw/Mn) and d′ (Mz/Mw) are determined by Gel Permeation Chromatography (GPC). The GPC was performed as follows.
Equipment: Agilent 1260 Series Degasser (Part Number G1322A): Agilent 1260 Series Isocratic Pump (Part Number G1310B); Agilent 1260 Series Autosampler (Part Number G1329B); Agilent 1260 Series Thermostatted Column Compartment (Part Number G1316A); Agilent 1260 Series Multiple Wavelength Detector (Part Number G1365C); and Agilent 1260 Series Refractive Index Detector (Part Number G1362A).
Column set: 1× Agilent ResiPore 50×4.6 mm Guard Column (Part Number PL1513-1300); and 2× Agilent ResiPore 250×4.6 mm 3 um Particle Size Columns (Part Number PL1513-5300).
Software: Cirrus 3.3, ChemStation B04.03 [52].
Solvent: THF Stabilized with 250 ppm BHT
Flow Rate: 0.45 ml/min.
Column Compartment Temperature: 40° C.
Injection Volume: 5 ml
Sample Preparation: Weighed approximately 0.06 grams of sample into a vial. Dissolve in 10 ml THF then filter through a 0.45 micron PTFE membrane. Run sample the same day it is prepared.
Polystyrene calibration standard used.

The molecular weight averages used in establishing molecular weight/property relationships are the number average (Mₙ), weight average (M_{w}) and z average (M_{z}) molecular weight. These averages are defined by the following expressions and are determined from the calculated Mᵢ:

$${M}_{n} = \frac{{\sum }_{i} {N}_{i} {M}_{i}}{{\sum }_{i} {N}_{i}} = \frac{{\sum }_{i} {W}_{i}}{{\sum }_{i} {W}_{i} / {M}_{i}} = \frac{{\sum }_{i} {h}_{i}}{{\sum }_{i} {h}_{i} / {M}_{i}}$$

$${M}_{w} = \frac{{\sum }_{i} {N}_{i} {M}_{i}^{2}}{{\sum }_{i} {N}_{i} {M}_{i}} = \frac{{\sum }_{i} {W}_{i} {M}_{i}}{{\sum }_{i} {M}_{i}} = \frac{{\sum }_{i} {h}_{i} {M}_{i}}{{\sum }_{i} {M}_{i}}$$

$${M}_{z} = \frac{{\sum }_{i} {N}_{i} {M}_{i}^{3}}{{\sum }_{i} {N}_{i} {M}_{i}^{2}} = \frac{{\sum }_{i} {W}_{i} {M}_{i}^{2}}{{\sum }_{i} {W}_{i} {M}_{i}} = \frac{{\sum }_{i} {h}_{i} {M}_{i}^{2}}{{\sum }_{i} {h}_{i} {M}_{i}}$$

Here Nᵢ and Wᵢ are the number and weight, respectively, of molecules having molecular weight Mᵢ.

The molecular weight distribution (MWD) is then calculated as Mw/Mn.

The crystallization temperature (Tc) and the glass temperature transition (Tg) were measured using differential scanning calorimetry (DSC), TA Instruments Q2000 equipped with a Liquid Nitrogen Cooling System (LNCS). The software was TA Instruments Universal Analysis 2000 Version 4.5A. We used a temperature ramp with a ramp rate of 10° C./min.

The particular features, structures, characteristics or embodiments may be combined in any suitable manner, as would be apparent to a person skilled in the art from this disclosure, in one or more embodiments.

### DETAILED DESCRIPTION OF THE DISCLOSURE

It has been found that polymerizing the polymerizable materials in pyrolysis oil and specifically plastic pyrolysis oil containing dienes using a Lewis acid, such as a Friedel-Crafts catalyst leads to the formation of a low molecular weight liquid resin with a low Tg, low viscosity, and some aromatic content. Other monomers such as branched mono-olefins, vinyl-aromatics, or other dienes may be combined with the pyrolysis oil to provide additional monomer feeds that can copolymerize to modify properties and compatibility and impart desired functionality to the resin.

The liquid resin comprises a low molecular weight pyrolysis oil-based homopolymer or copolymer resin obtained using a Friedel-Crafts catalyst and preferably has a low viscosity and some aromatic content.

The disclosure provides for an adhesive composition comprising an elastomer and one or more tackifying resins is remarkable in that one of the one or more tackifying resins comprises a liquid resin prepared from a liquified waste polymer, being preferably a pyrolysis oil via polymerization.

As it will be clear in the following, the liquid resin is an oligomeric product.

In a preferred embodiment, the adhesive composition elastomer is selected from the group consisting of styrene-isoprene block copolymers, polyacrylate resins, poly ethylene vinyl acetate (EVA) resins, polystyrene butadiene resins, random styrenebutadiene (SBR) copolymers, styrene-butadiene block copolymers, styrene-isoprene-butadiene-styrene (SIBS) copolymers, styrene-ethylene-propylene-styrene (SEPS) copolymers, styrene-ethylene-butylene-styrene (SEBS) block copolymers, amorphous poly-olefin (APO) resins, and mixtures thereof.

In some embodiments, the adhesive composition content of said elastomer ranges from 1 to 90 wt. %.

In some embodiments, the content of tackifying resin derived from liquid waste polymer ranges from 1 to 75 wt. %; preferably from 5 to 60 wt. %.

In some embodiments, the liquid resin (i.e., the oligomeric product) shows an olefinic content of at most 20.0 wt. % as determined by 1H NMR. With preference, the liquid resin has an olefinic content ranging from 0.1 to 20.0 wt. %; preferably from 0.3 to 15.0 wt. %; more preferably, from 0.5 to 10.0 wt. %. from 0.8 to 5.0 wt. %.

In some embodiments, the liquid resin (i.e., the oligomeric product) shows an aliphatic content ranging from 15 to 100 wt. % as determined by 1H NMR; preferably ranging from 40.0 to 99.5 wt. %; more preferably ranging from 50.0 to 99.0 wt. %; even more preferably ranging from 70.0 to 98.5 wt. %; and most preferably ranging from 80.0 to 98.0 wt. %.

In one or more embodiments, the liquid resin (i.e., the oligomeric product) shows an aromatic content ranging from 0.3 to 45.0 wt. % as determined by 1H NMR; preferably ranging from 0.5 to 40.0 wt. %; more preferably ranging from 0.8 to 30.0 wt. %; and even more preferably ranging from 1.0 to 20.0 wt. %.

In embodiments, the liquid resin (i.e., the oligomeric product) has a viscosity at 30° C. ranging from 10 to 50 000 cps measured using a Brookfield viscometer; preferably ranging from 10 to 50 000 cps; more ranging from 10 to 10 000 cps; even more preferably ranging from 10 to 4 000 cps; and most preferably ranging from 10 to 2 500 cps.

In some embodiments, the liquid resin (i.e., the oligomeric product) has a number average molecular weight (Mn) ranging from 100 to 10,000 g/mol measured using gel permeation chromatography and a polystyrene calibration; preferably from 120 to 5,000 g/mol; more preferably from 140 to 1,000 g/mol; even more preferably from 150 to 800 g/mol; most preferably from 180 to 700 g/mol; or from 200 to 650 g/mol; or from 250 to 550 g/mol.

In some embodiments, the liquid resin (i.e., the oligomeric product) has a weight average molecular weight (Mw) ranging from 100 to 10,000 g/mol measured using gel permeation chromatography and a polystyrene calibration; preferably from 120 to 5,000 g/mol; more preferably from 150 to 3,500 g/mol; and even more preferably from 200 to 2,000 g/mol.

In some embodiments, the liquid resin (i.e., the oligomeric product) has a z-average molecular weight (Mz) ranging from 100 to 100,000 g/mol measured using gel permeation chromatography and a polystyrene calibration; preferably from 120 to 85,000 g/mol; more preferably from 150 to 70,000 g/mol; and even more preferably from 200 to 50,000 g/mol.

In some embodiments, the liquid resin (i.e., the oligomeric product) has a Mw/Mn ranging from 1.0 to 10.0; preferably from 1.0 to 5.0; more preferably from 1.0 to 4.0; and even more preferably from 1.1 to 2.8.

In a preferred embodiment, the liquid resin (i.e., the oligomeric product) comprises a comonomer selected from the group consisting of styrene, alpha-methyl styrene, 2-methyl styrene, 3-methyl styrene, 4-methyl styrene, 4-t-butyl styrene, 4-cyclohexyl styrene, 4-dodecyl styrene, 2,4-diisopropyl styrene, 2.4.6-trimethyl styrene, 2-ethyl-4-benzylstyrene, 4-(phenylbutyl) styrene, 1-vinyl naphthalene, 2-vinyl naphthalene, vinyl anthracene, 4-methoxy styrene, monochlorostyrene, dichlorostyrene, divinyl benzene, Indene, methyl-Indene, and mixtures thereof; with preference the liquid resin (i.e., the oligomeric product) comprises a comonomer being or comprising styrene.

In embodiments, the liquid resin (i.e., the oligomeric product) comprises a comonomer present at a concentration ranging from 0.01 to 90 mol. % based on the total weight of the liquid resin, preferably from 1.0 to 80 mol. %.

In some embodiments, the liquid resin (i.e., the oligomeric product) is a wax and/or shows a crystallization temperature (Tc) below 35° C.; preferably ranging from 18 to 32° C.; more preferably ranging from 20 to 30° C.; and even more preferably ranging from 21 to 29° C.

In embodiments, the liquid resin (i.e., the oligomeric product) is a resin and/or shows a glass transition temperature (Tg) below −20° C. as determined by Differential Scanning calorimetry; preferably ranging from −60° C. to −25° C.; more preferably from −50° C. to −30° C.; and even more preferably from −49° C. to −35° C.

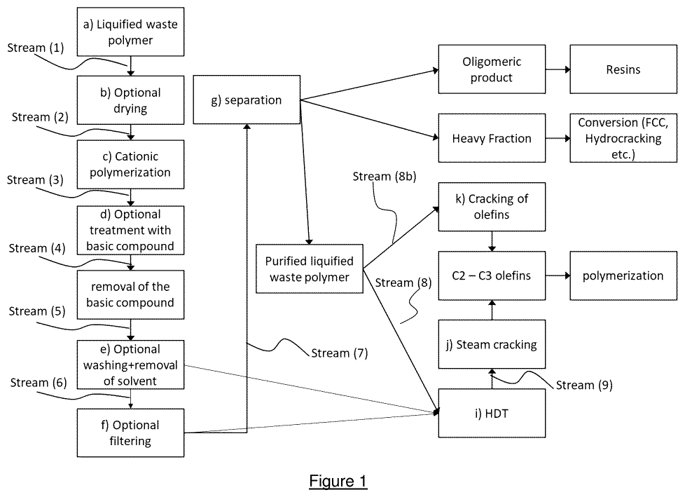

The disclosure also provides for a process and an installation for the production of said oligomeric product. Reference is made to FIG. 1 related to the process and FIG. 19 related to the installation. The installation and the process are described herein jointly.

The process for the preparation of a liquid resin from a liquified waste polymer, the liquid resin being an oligomeric product and being suitable to prepare an adhesive composition according to the disclosure comprises the following steps:
a) providing a feedstream 1 containing liquified waste polymer, wherein said feedstream 1 contains at least 0.1 wt. % to at most 80 wt. % of dienes based on the total weight of said feedstream 1;
c) performing a polymerization reaction on said feedstream 1 under polymerization conditions to obtain a first product stream 3 comprising an oligomeric product;
d) optionally, performing a neutralization reaction by contacting said first product stream 3 with a basic compound to obtain a neutralized product stream 4 and removing said basic compound from neutralized product stream 4 to obtain a second product stream 5;
g) performing a separation to separate the oligomeric product from the purified liquified waste polymer.

In preferred embodiments illustrated in FIG. 1, the process for the preparation of a liquid resin from a liquified waste polymer, the liquid resin being an oligomeric product and being suitable to prepare an adhesive composition comprises the following steps:
a) providing a feedstream 1 containing liquified waste polymer, wherein said feedstream 1 contains at least 0.1 wt. % to at most 80 wt. % of dienes based on the total weight of said feedstream 1;
b) optionally, drying the feedstream 1 to obtain a dried feedstream 2;
c) performing a polymerization reaction on said feedstream 1 or on the dried feedstream 2 under polymerization conditions to obtain a first product stream 3 comprising an oligomeric product;
d) optionally, performing a neutralization reaction by contacting said first product stream 3 with a basic compound to obtain a neutralized product stream 4 and removing said basic compound from neutralized product stream 4 to obtain a second product stream 5;
e) optionally, washing the first product stream 3 or the second product stream 5 with a solvent to obtain a washed stream 6;
f) optionally, filtering the stream obtained in the previous step to obtain a filtered stream 7 wherein the filtering is performed to remove solids from the first product stream or from the second product stream 5 or from the washed stream 6, and/or to coalesce remaining traces of solvent if any;
g) performing a separation to separate the oligomeric product from the purified liquified waste polymer.

In a preferred embodiment, the process for the preparation of a liquid resin from a liquified waste polymer, the liquid resin being an oligomeric product and being suitable to prepare an adhesive composition comprises the following steps:
a) providing a feedstream 1 containing liquified waste polymer, wherein said feedstream 1 contains at least 0.1 wt. % to at most 80 wt. % of dienes based on the total weight of said feedstream 1;
b) drying the feedstream 1 to obtain a dried feedstream 2;
c) performing a cationic polymerization reaction on the dried feedstream 2 under polymerization conditions to obtain a first product stream 3 comprising an oligomeric product;
d) performing a neutralization reaction by contacting said first product stream 3 with a basic compound to obtain a neutralized product stream 4 and removing said basic compound from neutralized product stream 4 to obtain a second product stream 5;
e) washing the second product stream 5 with a solvent to obtain a washed stream 6;
f) filtering the washed stream 6 to obtain a filtered stream 7 wherein the filtering is performed to remove solids from the washed stream 6, and/or to coalesce remaining traces of solvent if any;
g) performing a separation to separate the oligomeric product from the purified liquified waste polymer.

The disclosure also provides an installation 100 for carrying out a process for the preparation of a liquid resin from a liquified waste polymer, the liquid resin being an oligomeric product and being suitable to prepare an adhesive composition, said installation is remarkable in that it comprises:
a polymerization section C comprising one or more polymerization reactors 23;
an optional neutralization section D;
a separation section comprising a separation unit 32 comprising one or more distillation columns.
   wherein the polymerization section C and the separation section are fluidically connected in series, and wherein the neutralization section D when present is placed downstream of the polymerization section and upstream of the separation section.

In some embodiments, the disclosure also provides an installation 100 for carrying out a process for the preparation of a liquid resin from a liquified waste polymer, the liquid resin being an oligomeric product and being suitable to prepare an adhesive composition, said installation is remarkable in that it comprises, fluidically connected in the given order:
an optional pre-treatment section A;
an optional drying section B;
a polymerization section C comprising one or more polymerization reactors 23;
an optional neutralization section D;
an optional washing section E;
an optional filtering section; and
a separation section comprising a separation unit 32 comprising one or more distillation columns.

In an embodiment, the disclosure also provides an installation 100 for carrying out a process for the preparation of a liquid resin from a liquified waste polymer, the liquid resin being an oligomeric product and being suitable to prepare an adhesive composition, said installation is remarkable in that it comprises:
a pre-treatment section A comprising a pyrolysis unit 11, one or more separation units (13, 16, 19) and an optional washing unit 18;
a drying section B comprising a decanter and/or a centrifuge 21; and/or a molecular sieve 22;
a polymerization section C comprising one or more polymerization reactors 23; preferably loaded with an acidic catalyst;
a neutralization section D a mixing reactor 24 and an optional separation unit 26 comprising at least one selected from a decanter, a centrifuge, and a filter;
a washing section E comprising a mixing vessel 28 and a separation unit 30 comprising a decanter and/or a centrifuge;
a filtering section comprising a filter and an optional molecular sieve 31; and
a separation section comprising a separation unit 32 comprising one or more distillation columns.

### The Feedstream 1 and Step a) of Providing a Feedstream 1 and the Pretreatment Section A

The feedstream 1 used in the process contains a liquified waste polymer.

It is preferred that the liquified waste polymer is originated from the pyrolysis of plastic waste 10, so that the liquified waste polymer is a pyrolysis plastic oil.

Thus, in a preferred embodiment, step a) of providing a feedstream 1 comprises the preliminary steps of preparation of liquified waste polymer including:
a1) providing a waste stream 10 being preferably a waste plastics stream;
a2) pyrolyzing said waste stream 10 at a temperature of at least 200° C.;
a3) recovering a pyrolizer effluent 12 and separating said pyrolizer effluent into a C1 to C4 hydrocarbons fraction 14, a fraction 17 having a boiling range higher than 350° C. and a remaining fraction being said pyrolysis plastic oil.

In such cases, the installation 100 may comprise a pretreatment section A comprising a pyrolysis unit 11, one or more separation units (13, 16, 19) and an optional washing unit 18. With preference, the pretreatment section A comprises one or more separation units selected from:
a first separation unit 13 to remove pyrolysis gas comprising a gas-liquid separator
a second separation unit 16 to remove the fraction having a boiling range higher than 350° C. comprising one or more distillation columns; and
a third separation unit 19 to remove particles and gums, comprising a filter and/or a centrifuge

In some embodiments (not illustrated) the process further comprises a further preliminary step of treatment of the fraction having a boiling range higher than 350° C. and therefore further comprises a step a4) of sending said fraction having a boiling range higher than 350° C. into a Fluid Catalytic Cracking (FCC) unit, or a hydrocracking unit, a coker or a visbreaker; or of blending said fraction having a boiling range higher than 350° C. in crude oil or in a crude oil cut to be further refined.

In a preferred embodiment, the liquified waste plastics comprise mainly pyrolysis plastic oil. Pyrolysis plastic oil is produced from plastic wastes that are pyrolyzed. An example of a pyrolysis process for waste plastics is disclosed in U.S. Pat. No. 8,895,790 or in US 2014/228606 and in WO2016/009,333.

In a waste plastics pyrolyzer, mixed plastics (e.g., waste plastics 10) are placed in pyrolysis unit or pyrolyzer. In the pyrolysis unit 11, the waste plastics is converted via pyrolysis to a pyrolysis product, wherein the pyrolysis product 12 comprises a gas phase (e.g., pyrolysis gases, such as C1 to C4 gases, hydrogen (H₂), carbon monoxide (CO), carbon dioxide (CO₂) mainly) and a liquid phase being pyrolysis plastic oil.

The plastic waste 10 may include post-consumer waste plastics, such as mixed plastic waste. Mixed plastics can comprise non-chlorinated plastics (e.g., polyolefins, polyethylene, polypropylene, polystyrene, copolymers, etc.), chlorinated plastics (e.g., polyvinylchloride (PVC), polyvinylidene chloride (PVDC), etc.), and the like, or mixtures thereof. Generally, waste plastics 10 comprise long-chain molecules or polymer hydrocarbons. Waste tires may also be used. Namely waste plastics may also include used tires.

The pyrolysis unit 11 may be any suitable vessel configured to convert waste plastics into gas phase and liquid phase products (e.g., simultaneously). The vessel may be configured for gas phase, liquid phase, vapor-liquid phase, gas-solid phase, liquid-solid phase, or slurry phase operation.

The vessel may contain one or more beds of inert material or pyrolysis catalyst comprising sand, zeolite, alumina, a catalytic cracking catalyst, or combinations thereof. Generally, the pyrolysis catalyst is capable of transferring heat to the components subjected to the pyrolysis process in the pyrolysis unit. Alternatively, the pyrolysis unit 11 can be operated without any catalyst (e.g., pure thermal pyrolysis).

The pyrolysis unit 11 may be operated adiabatically, isothermally, nonadiabatically, non-isothermally, or combinations thereof. The pyrolysis reactions of this disclosure may be carried out in a single stage or in multiple stages. For example, the pyrolysis unit 11 can be two reactor vessels fluidly connected in series.

In a configuration (not illustrated) where the pyrolysis unit comprises two vessels, the pyrolysis process may be divided into a first stage which is performed in a first vessel, and in a second stage fluidly connected downstream to the first stage which is performed in the second vessel. As will be appreciated by one of skill in the art, and with the help of this disclosure, the second stage may enhance the pyrolysis of an intermediate pyrolysis product stream flowing from the first stage into the second stage, to yield a pyrolysis product flowing from the second stage. In some configurations, the first stage may utilize thermal cracking of the waste plastics, and the second stage may utilize thermal or catalytic cracking of the waste plastics to yield the pyrolysis product flowing from the second stage. Alternatively, the first stage may utilize catalytic cracking of the waste plastics, and the second stage may utilize thermal or catalytic cracking of the waste plastics to yield the pyrolysis product flowing from the second stage.

In some configurations, the pyrolysis unit 11 may include one or more equipment configured to convert mixed plastics into gas phase and liquid phase products. The one or more equipment may or may not contain an inert material or pyrolysis catalyst as described above. Examples of such equipment include one or more of heated extruders, heated rotating kiln, heated tank-type reactors, packed bed reactors, bubbling fluidized bed reactors, circulating fluidized bed reactors, empty heated vessels, enclosed heated surfaces where plastic flows down along the wall and cracks, vessels surrounded by ovens or furnaces, or any other suitable equipment offering a heated surface to assist in cracking.

The pyrolysis unit 11 can be configured to pyrolyze (e.g., crack), and in some aspects (e.g., where hydrogen is added to the pyrolysis unit), additionally hydrogenate components of the waste plastics stream fed to the pyrolysis unit. Examples of reactions that may occur in the pyrolysis unit 11 include, but are not limited to isomerization of one or more normal paraffins to one or more i-paraffins, selective ring opening of one or more cycloparaffins to one or more i-paraffins, cracking of long chain length molecules to short chain length molecules, removal of heteroatoms from heteroatom-containing hydrocarbons (e.g., dechlorination), hydrogenation of coke generated in the process, or combinations thereof.

In one or more configurations of the pyrolysis unit 11, a headspace purge gas can be utilized in all or a portion of the pyrolysis stage(s) (conversion of waste plastics to a liquid phase and/or gas phase products) to enhance cracking of plastics, produce valuable products, provide a feed for steam cracking, or combinations thereof. The headspace purge gas may include hydrogen (H2), C1 to C4 hydrocarbon gases (e.g., alkanes, methane, ethane, propane, butane, isobutane), inert gases (e.g., nitrogen (N₂), argon, helium, steam), and the like, or combinations thereof. The use of a headspace purge gas assists in the dechlorination in the pyrolysis unit, when the waste plastics comprise chlorinated plastics. The headspace purge gas may be introduced to the pyrolysis unit to aid in the removal of volatiles entrained in the melted mixed plastics present in the pyrolysis unit.

A hydrogen (H₂) containing stream can be added to the pyrolysis unit 11 to enrich the pyrolysis unit environment with H₂, assist in stripping entrapped hydrogen chloride in the pyrolysis unit, provide a local environment rich in hydrogen in the pyrolysis melt or liquid, or combinations thereof; for example, via an H₂ containing stream fed directly to the pyrolysis unit 11 independently of the waste plastics 10 stream. In some aspects, H₂ can also be introduced along with stream to the pyrolysis unit 11, with adequate safety measures incorporated for hydrogen handling with plastics feed.

The pyrolysis unit 11 may facilitate any reaction of the components of the waste plastics 10 stream in the presence of, or with, hydrogen. Reactions may occur such as the addition of hydrogen atoms to double bonds of unsaturated molecules (e.g., olefins), resulting in saturated molecules (e.g., paraffins, i-paraffins, naphthenes). Additionally, or alternatively, reactions in the pyrolysis unit may cause a rupture of a bond of an organic compound, with a subsequent reaction and/or replacement of a heteroatom with hydrogen.

The use of hydrogen in the pyrolysis unit 11 can have beneficial effects of (I) reducing the coke as a result of cracking, (II) keeping the catalyst used (if any) in the process in an active condition, (III) improving removal of chloride from stream such that the pyrolysis product from pyrolysis unit 11 is substantially dechlorinated with respect to waste plastics stream, which minimizes the chloride removal requirement in units downstream of the pyrolysis unit, (IV) hydrogenating of olefins, (V) reducing diolefins in pyrolysis product, (VI) helping operate the pyrolysis unit 11 at reduced temperatures for same levels of conversion of waste plastics 10 stream in the pyrolysis unit, or combinations of (I)-(VI).

The pyrolysis processes in the pyrolysis unit 11 may be low-severity or high-severity.

Low-severity pyrolysis processes may occur at a temperature of less than about 450° C.; for example, at a temperature ranging from 250° C. to 450° C., preferably from 275° C. to 425° C., more preferably 300° C. to 400° C., and may produce pyrolysis oils rich in mono- and di-olefins as well as a significant amount of aromatics.

High-severity pyrolysis processes may occur at a temperature of equal to or greater than about 450° C.; for example, at a temperature ranging from 450° C. to 750° C., preferably from 500° C. to 700° C., more preferably from 550° C. to 650° C., and may produce pyrolysis oils rich in aromatics, as well as more gas products (as compared with low severity pyrolysis).

As will be appreciated by one of skill in the art, a high severity pyrolysis process will lead to the formation of more olefins and diolefins. Those olefins and diolefins cannot easily be recovered.

A pyrolysis product 12 can be recovered as an effluent from the pyrolysis unit 11 and conveyed (e.g., flowed, for example via pumping, gravity, pressure differential, etc.) to a pyrolysis separating unit (i.e. a first separation unit 13). The pyrolysis product 12 can be separated in the pyrolysis separating unit into a pyrolysis gas 14 stream and a pyrolysis plastic oil further used in step a) of the present disclosure.

The first separation unit 13 may comprise any suitable gas-liquid separator, such as a vapor-liquid separator, oil-gas separators, gas-liquid separators, degassers, scrubbers, traps, flash drams, compressor suction drams, gravity separators, centrifugal separators, filter vane separators, mist eliminator pads, liquid-gas coalescers, distillation columns, and the like, or combinations thereof.

In a preferred embodiment, said of pyrolysis plastic oil originates directly from the pyrolysis of plastic wastes without further chemical transformation or separation.

In a preferred embodiment, the feedstream 1 contains only liquified waste polymer even more preferably only pyrolysis plastic oil. Alternatively, the feedstream 1 contains at least 10 wt. % of liquified waste polymer based on the total weight of the feedstream 1; preferably at least 25 wt. %, more preferably at least 50 wt. %, even more preferably at least 75 wt. %.

The feedstream 1 may contain 100 wt. % of said liquified waste polymer based on the total weight of the feedstream 1; preferably at most 95 wt. %; more preferably, at most 90 wt. %; even more preferably at most 85 wt. %, and most preferably at most 80 wt. %. When the feedstream 1 contains less than 100 wt. % of liquid waste polymer the rest can be for instance a diluent or other hydrocarbons.

In a preferred embodiment, the feedstream 1 presents a bromine number of at most 150 g Br₂/100 g as measured according to ASTM D1159-07(2017), preferably at most 100 g Br₂/100 g; more preferably at most 80 g Br₂/100 g, and even more preferably, at most 50 g Br₂/100 g.

In a preferred embodiment, the liquified waste polymer in the feedstream 1 has a starting boiling point of at least 15° C. and a final boiling point of at most 700° C. With preference, the final boiling point is at most 600° C.; more preferably 560° C., even more preferably 450° C. even more preferably 350° C., the most preferred 250° C.

In some embodiment, step a) of providing a providing a feedstream 1 comprises one or more sub-steps of pre-treatment of the liquified waste polymer.

Indeed, depending on the origin of the liquified waste polymer, some pre-treatment may be necessary. Namely, before purifying the liquified waste polymer in the inventive process, the following pre-treatment may be considered either on the liquified waste polymer or on the feedstream 1.

In a preferred embodiment, the liquified waste polymer or the feedstream 1 is pretreated in a washing or a desalting unit 18 to remove water-soluble salts before being treated in the inventive process.

In another preferred embodiment, the liquified waste polymer or the feedstream 1 is pretreated in a separation unit 19 to extract the particles and gums 20 by filtration, centrifugation, or a combination of the two technics before being treated in the inventive process.

### The Optional Step b) of Drying the Feedstream 1 to Obtain a Dried Feedstream 2 and the Drying Section B

In another preferred embodiment, the liquified waste polymer or the feedstream 1 is pretreated in a dewatering unit (i.e., a drying unit) to remove water in said hydrocarbon stream to reach a water content of less than 0.1% vol preferably of less than 0.05% vol according to ASTM D95-13(2018) before being treated in the inventive process, even more preferably said liquified waste polymer may be decanted.

Preferably, the feedstream 1 is dried before being sent to the polymerization unit. step b) of drying can be performed a sub-step b1) of decantation and/or centrifugation; with preference, the sub-step b1) is followed by a sub-step b2) of drying using a molecular sieve 22 to reach a water content of less than 0.1 vol. % according to ASTM D95-13 (2018).

In such cases, the installation 100 may comprise a drying section B comprising:
a decanter and/or a centrifuge 21; and/or
a molecular sieve 22

### Step c) of Performing a Polymerization Reaction on the Feedstream 1 or the Dried Feedstream 2 and the Polymerization Section C

Step c) comprises performing a polymerization reaction being a cationic polymerization or a free radical polymerization or an anionic polymerization; with preference, the polymerization reaction is a cationic polymerization. Thus, the polymerization section C comprises one or more polymerization reactors 23 loaded with a catalyst; preferably an acidic catalyst.

In an embodiment, said polymerization is an anionic polymerization. In such case, said anionic polymerization is performed using an organolithium containing anionic initiator and terminated by reaction with a proton donating termination agent selected to form the lithium salt which can be filtered.

In the embodiments wherein the polymerization reaction is a cationic polymerization, the installation comprises a polymerization section C comprising one or more polymerization reactors 23 loaded with an acidic catalyst.

In an embodiment, said polymerization is a free radical polymerization. In such case, said free radical polymerization is performed with an initiator utilized to generate an initial radical leading to monomer propagation followed by a termination via combination or disproportionation.

In preferred embodiments, the polymerization reaction is a cationic polymerization. Thus, preferably, step c) is performed in the presence of an acidic catalyst; with preference, the acidic catalyst is a Brönsted acid or a Lewis acid; preferably a Lewis acid in the form of a transition metal halogenate.

Friedel-Crafts catalysts are strong Lewis acid catalysts that polymerize monomers via a cationic polymerization reaction mechanism. According to various embodiments of the present invention, examples of Friedel-Crafts catalysts that may be used to polymerize a monomer feed comprising pyrolysis oil include, but are not limited to, boron trifluoride, aluminum trichloride, tin tetrachloride, titanium trichloride, titanium tetrachloride, iron chloride (III), aluminum tri-bromide, dichloromono ethyl aluminum, and complexes thereof, such as boron trifluoride-phenol complex, boron trifluoride-ethanol complex, boron trifluoride-ether complex and the like, especially, boron trifluoride-phenol complex and boron trifluoride-ethanol complex. The Friedel-Craft catalyst may also include a liquid aluminum chloride/hydrochloric acid/substituted aromatics complex, the aromatic being for example o-xylene, mesitylene, ethyl benzene, isopropyl benzene, and the like, such as short or long-chain alkylbenzenes. The alky chain may be linear or branched and may vary from 2 to 30 carbon atoms. Acidic liquid AlCl, obtained as by-products during the alkylation of benzene or any other substituted aromatics (toluene, xylenes) with branched-chain olefins may also be used. The branched-chain olefins may be produced via the boron trifluoride oligomerization of propylene and fractionation (e. g. C12 olefins or C24 olefins may be alkylated with aromatics).

The Friedel—Crafts catalysts most preferred are AlCl₃ and BF₃.

Thus, with preference, step c) is performed in the presence of an acidic catalyst being a Lewis acid chosen among BF₃, complexes of boron trifluoride, AlCl₃, SnCl₄, ZnCl₂, FeCl₃ and TiCl₃, alkyl aluminum chlorides, H₂SO₄ or any mixture thereof. It is understood that BF₃ refers to BF₃ in the gaseous form. A particularly suitable complex of boron trifluoride is boron trifluoride etherate.

In an embodiment, step c) is performed in the presence of an acidic catalyst being a Lewis acid with an acidic catalyst concentration ranging from 0.5 wt. % to 5.0 wt. % based on the total weight of said feedstream 1,

In an embodiment, the reaction conditions include a contact time of at least 5 min to at most 5 hours. Preferably, step c) is performed under an inert atmosphere, preferably under N₂ atmosphere.

The resins described herein may be prepared by a continuous solution polymerization process wherein the Friedel-Craft catalyst, optional comonomers, and a suitable aromatic or aliphatic hydrocarbon solvent, such as, for example, toluene, xylene, or heptane, are continuously added to a reactor vessel to form the desired homopolymer or copolymer. Alternatively, the pyrolysis oil-based polymers may be prepared through a batch process in which all the initiator, monomers, and solvent are combined in the reactor together and mixed. The acid catalyst may also be a solid catalyst.

Preferably, during a continuous polymerization reaction, the mass flow rate of the catalyst feed is from 0.01 to 20 wt. % based on the total mass flow rate of monomer feed and catalyst feed; preferably, from 0.1 to 5 wt. %, and more preferably from 0.1 to 3 wt. %.

The reaction temperature of the mixture in the reactor vessel is also preferably maintained at a temperature of about-10 to 50° C., when using BF, as the Friedel-Crafts catalyst and about 10 to 60° C., when using AlCl₃.

The low molecular weight pyrolysis oil-based homopolymers or copolymers resins according to the embodiments of the present invention are liquid resins with a number average molecular weight between 100 and 10,000 g/mol and more preferably 250 and 4,000 g/mol, as measured via gel permeation chromatography and converted using polystyrene calibration.

In a preferred embodiment, the cationic polymerization is performed in the presence of an acidic catalyst, being preferably chosen among BF₃, AlCl₃, SnCl₄, and TiCl₃, ion exchange resin, Cl₂AlEt, H₂SO₄ or any mixture thereof.

In an embodiment, at least one of AlCl₃, and BF₃, may be used to polymerize a monomer feed comprising pyrolysis oil and a comonomer in which the comonomer content of the monomer feed is <90 wt. %, <40 wt. %, <25 wt. %, or <15 wt. %, based on the total moles of the monomer in the monomer feed. Preferably, the polymerization initiator is BF₃.

In particular, the acidic catalyst can be a solid acidic catalyst. The preferred solid acid catalyst includes molecular sieves such as zeolite or silicalite. The use of a solid acidic catalyst is particularly advantageous. The solid acid catalyst can be easily separated. When a solid acid catalyst is used, the use of a neutralization step d) and step of washing e) may not be required.

In an embodiment, step c) is performed with a solid acid catalyst being a molecular sieve, preferably selected among the topologies MFI, BEA, FAU, MEL, and the polymerization conditions comprise a Liquid Hourly Space Velocity (LHSV) ranging from 0.5 to 5.0 h⁻¹. In a preferred embodiment the molecular sieve contains one or more heteroatoms selected from Ti, Sn, Mo, W, Fe, Ni, Co, Cu, Zr, Hf, Nb, and Ta.

In an embodiment, step c) is performed with a solid acid catalyst being an ion exchange resin, preferably a sulfonic ion-exchange resin such as Amberlyst A15 or A35 and the polymerization conditions comprise an LHSV ranging from 0.5 to 5.0 h⁻¹. Ion exchange resin includes macro-porous polymeric resins having acidic properties for instance having the capability to exchange H⁺ ions. In particular, ion exchange resins can comprise styrene-divinylbenzene copolymers such as for instance Amberlyst resins. In a preferred embodiment, the ion exchange resin is an Amberlyst 15.

In an embodiment, step c) is performed with a solid acid catalyst being a clay or an activated clay and the polymerization conditions comprise an LHSV ranging from 0.5 to 5.0 h⁻¹.

### The Optional Neutralization Step d) and the Neutralization Section D

When an acidic catalyst soluble is used in step c) to produce the first product stream 3, it may be required to perform a neutralization step d1) preferably followed by a washing step d2).

Thus, in some embodiments, the installation 100 comprises a neutralization section D comprising a mixing reactor 24 and an optional separation unit 26 comprising at least one selected from a decanter, a centrifuge, and a filter.

Step d) is a neutralization step that can be done by adding a base directly on said first product stream 3. In that case, a basic compound, generally solid is put in contact with said first product stream 3, the mixture obtained is then agitated for at least 1 second to at most 24 h, preferably during at least 1 minute to at most 12 h. After agitation, the remaining base in the form of a solid is separated from the liquid, said liquid being said second product stream 5.

The neutralization step can also be done by washing said first product stream 3 with a basic compound 25 being a basic solution. Basic aqueous solutions are preferred, basic aqueous solutions containing NaOH and/or NH4OH are the most preferred type of solution. The basic solutions are put in contact and agitated for at least 1 second to at most 24 h, preferably for at least 1 minute to at most 12 h. Then the mixture is let for separation to remove the second product stream 5 (being the organic phase) from the aqueous phase. The separation can consist for instance in a decantation. The decantation is known per se, it consists in letting the immiscible organic and aqueous phases being separated with the help of gravity.

The neutralization step can also be done by putting simultaneously or successively one or more basic compound 25, preferably a base containing alkali/alkaline earth metals, more preferably a solid base such as LiOH, NaOH, CsOH, Ba(OH) 2, Na₂O, KOH, K₂O, CaO, Ca(OH)₂, MgO, Mg(OH)₂ or any mixture thereof or any strong base alone or in mixture, and a basic aqueous solution in contact with said first product stream 3. After agitation of at least 1 second to at most 24 h, the solid, the organic phase being said second product stream 5, and the aqueous phase are separated.

In a more preferred embodiment, step d) is performed in continuous mode, namely addition of fresh basic compound is performed on the first product stream 3 at the entry of for instance a mixing reactor 24 while simultaneously, at the exit of the mixing reactor 24 the spent basic compound is removed to obtain the second product stream 5.

In a preferred embodiment, the basic compound comprises an alkali metal cation, alkaline earth metal cation, and ammonium quaternary base, being preferably chosen among TMAOH, TEAOH, TBAOH, TPAOH, or any mixture thereof.

### The Optional Washing Step e) and the Washing Section E

The washing step e) comprises washing the first product stream 3 or the second product stream 5 with a solvent 27 to obtain a washed stream 6. In some embodiments, the washing step e) is performed after the neutralization step d) to remove traces of the basic compound 25. In such a case, the washing step is performed with a ratio of said second product stream 5, or optionally of said neutralized product stream 4, over said solvent 27 ranging from 0.05 to 20.

For example, the washing is performed at a temperature ranging from 5° C. to 95° C., preferably under atmospheric pressure. The washing can be performed in continuous mode or in batch mode.

In a preferred embodiment, the solvent is selected water or an acidic water solution, said solution comprising one or more organic acids like for instance citric acid (C₆H₈O₇), formic acid (CH₂O₂), acetic acid (CH₃COOH), sulfamic acid (H₃NSO₃) or inorganic acids being hydrochloric acid (HCl), nitric acid (HNO₃), sulfuric acid (H₂SO₄), phosphoric acid (H₃PO₄) and any combination thereof.

For example, the acidic solution has a pH ranging from 0.1 to 6.9.

The washing is preferably performed until the pH of said washed stream 6, or optionally on said second product stream 5, or neutralized product stream 4, is in the range of 5.0 to 9.0, preferably in the range of 6.0 to 8.0, even more preferably in the range of 6.5 to 7.5.

The washing is preferably followed by a decantation and/or a centrifugation to separate the solvent 27 from the washed mixture 29, to produce a washed stream 6.

In such cases, the installation 100 may comprise a washing section E comprising a mixing vessel 28 and a separation unit 30 comprising a decanter and/or a centrifuge.

### The Optional Filtering Step f) and the Filtering Section

The process can comprise a filtering step f) comprising filtering the stream obtained in the previous step to obtain a filtered stream 7 wherein the filtering is performed to remove solids.

In the embodiments wherein the preceding step was the washing step e), the process may comprise a filtering step f) comprising filtering the washed stream 6 (i.e., the stream obtained in the previous step) to obtain a filtered stream 7 wherein the filtering is performed to remove solids from the washed stream 6, and/or to coalesce remaining traces of solvent if any.

In a preferred embodiment, the filtering comprises a first sub-step of filtering to remove solids and/or to coalesce remaining traces of solvent if any, preferably followed by a second sub-step of dewatering preferably using a molecular sieve to reach a water content of less than 0.1 vol. % preferably of less than 0.05% vol according to ASTM D95-13 (2018) on the filtered stream 7.

In such cases, the installation 100 may comprise a filtering section comprising a filter (not represented) and an optional molecular sieve 31.

### The Separation Step g) and the Separation Section

The process comprises a step g) of performing a separation to separate the oligomeric product 33 from the purified liquified waste polymer 34.

In a preferred embodiment said step g) of separation is performed via distillation or steam distillation or vacuum stripping or fractional distillation or any combination.

Thus, the installation comprises a separation unit 32 comprising one or more distillation columns.

The oligomeric product 33 obtained can also be used further converted into valuable as valuable resins. After the separation step g), the oligomeric product 33 may be recovered and mixed with an elastomer, a curing agent, and a filler to obtain a rubber composition or is used as a tackifying resin and mixed with an elastomer to form an adhesive composition.

If not yet done in a pretreatment section, the process may comprise a step of separating and recovering a fraction having a boiling range higher than 350° C., preferably higher than 300° C. This fraction can be recovered from any of the purified liquified waste polymer 34, the filtered stream 7, the washed stream 6, the second product stream 5, the neutralized product stream 4, the first product stream 3, the dried feedstream, or the feed stream 1. The fraction having an initial boiling point of at least 350° C., preferably at least 300° C., can therefore be removed and sent into an FCC unit, or a hydrocracking unit, a coker or a visbreaker or blending said fraction having a boiling range higher than 350° C. in crude oil or in a crude oil cut to be further refined.

With preference, after the separation step g), the purified liquified waste polymer 34 is recovered and blended in the fuel pool; with preference, the purified liquified waste polymer is separated in a naphtha cut having a boiling range of less than 150° C. and a diesel cut having a boiling range between 15° and 350° C., wherein said naphtha cut is incorporated in a naphtha pool, said diesel cut is incorporated in a diesel pool.

### The Production of an Adhesive Composition

The disclosure also provides a method to produce an adhesive composition comprising the production of an oligomeric product according to the process of the fifth aspect, and mixing the recovered oligomeric product with an elastomer to form an adhesive composition; wherein the oligomeric product is a liquid resin prepared from a liquified waste polymer and used as a tackifying resin.

### Detailed Description of the Adhesive Composition

The present invention, in a preferred embodiment, encompasses an adhesive composition comprising an elastomer, tackifying resin(s), remarkable in that at least one of said tackifying resin behaves as a processing oil. Hence there is no need to add a processing oil. More preferably, said adhesive composition do not contain any processing oil.

According to another preferred embodiments, the elastomer of the adhesive composition of the present invention may be selected from the group consisting of styrene-isoprene block copolymers, polyacrylate resins, poly ethylene vinyl acetate (EVA) resins, polystyrene butadiene resins, random styrenebutadiene (SBR) copolymers, styrene-butadiene block copolymers, styrene-isoprene-butadiene-styrene (SIBS), styrene-ethylene-propylene-styrene (SEPS), styrene-ethylene-butylene-styrene (SEBS) block copolymers, amorphous polyolefin (APO) resins, and mixtures thereof.

The adhesive compositions according to various embodiments of the present invention may also include a processing oil. Examples of processing oils include, but are not limited to, paraffinic, aliphatic-naphthenic aromatic resins, polyethylene glycol, petroleum oils, ester plasticizers, vulcanized vegetable oils, pine tar, phenolic resins, petroleum resins, polymeric esters and rosins. A processing oil may be used in an amount ranging from about 0 to about 50 wt. % based on the total weight of adhesive resin in the adhesive composition with a range of from about 5 to 35 wt. % being preferred. In a preferred embodiment, the inventive tackifying resin functions as a suitable replacement for the processing oil.

The tackifying resins according to the present invention may be incorporated into an adhesive composition by any method known to those having skill in the art. For example, one or more tackifying resins may be added during the compounding of the adhesive composition.

An exemplary conventional hot melt adhesive includes a styrene-isoprene-styrene (SIS) block copolymer, a hydrocarbon (C₅ or C₅/C₉) tackifying resin, a rosin ester tackifier, and/or a process oil. A tackifying resin derived from the liquid resin obtained via polymerization of the pyrolysis oil product may be used to replace all or part of the processing oil and used in conjunction with C5 or C5/C9 tackifying resin or rosin ester tackifier in this exemplary formulation. A SIS block copolymer may be employed in the hot melt adhesive, such as that which is sold by Kraton Performance Polymers Inc. of Houston, Tex. under the trade name Kraton. A C5 hydrocarbon tackifying resin may be utilized, such as that which is sold by Total Petrochemicals and Refining, Inc. of Houston, Tex. under the trade name Wingtack. A naphthenic process oil may be used in the hot melt adhesive as well, such as the one sold under the trade name Nyflex by Nynas AB of Stockholm, Sweden.

As is known to one skilled in the art, conventional hot melt adhesives may include a variety of other components including, but not limited to, starches, waxes, plasticizers, anti-oxidants, stabilizers, pigments, dyes, biocides, flame retardants, antistatic agents, or fillers. For example, the hot melt adhesive may include Ethanox 310, an antioxidant sold by Albemarle Corporation of Baton Rouge, La.

The components of an adhesive composition according to the present invention may be combined by any process known to one skilled in the art. For example, they may be introduced separately from each other and separate from, or in combination with, any of the individual components of the conventional adhesive composition. As a further example, a tackifying resin may be introduced to a composition comprising a styrene-isoprene-styrene (SIS) block copolymer and optionally, a hydrocarbon (C5-C9) tackifying resin. The process oil or process oil replacement, such as the liquid resin or oligomeric product from pyrolysis oil, may be added last. The adhesive composition may be mixed with a sigma blade mixer, for example, until it is homogeneous. Other methods of mixing include, but are not limited to, single or twin screws, and conventional tanks equipped with mixers like an anchor or turbine.

### EXAMPLES

### Method Used for Measuring the Diene Content

Unless otherwise specified, the following method was used to determine the content of dienes in the pyrolysis plastic oils in all the examples.

The determination of the diene content is performed with the help of the GPC with a UV detector. A given quantity of maleic anhydride is added in the pyrolysis plastic oil and let to react with the dienes via the Diels-Alder reaction. The reduction of the UV active MA peak in the chromatograph is monitored and used to determine diene concentration. In a first step, a calibration curve is prepared. The equipment used are

Agilent 1260 Series Degasser (Part Number G1322A); Agilent 1260 Series Isocratic Pump (Part Number G1310B); Agilent 1260 Series Autosampler (Part Number G1329B); Agilent 1260 Series Thermostatted Column Compartment (Part Number G1316A); Agilent 1260 Series Multiple Wavelength Detector (Part Number G1365C); and Agilent 1260 Series Refractive Index Detector (Part Number G1362A).

Column set: 1× Agilent ResiPore 50×4.6 mm Guard Column (Part Number PL1513-1300); and 2× Agilent ResiPore 250×4.6 mm 3 um Particle Size Columns (Part Number PL1513-5300).

Software: Cirrus 3.3, ChemStation B04.03 [52].

Solvent: THF Stabilized with 250 ppm BHT Flow Rate: 0.45 ml/min.
Column Compartment Temperature: 40° C.
Calibration: polystyrene standards from Agilent EasiCal Standards

### i) GPC-UV Calibration of Maleic Anhydride:

Samples of known concentration of maleic anhydride (MA) in THF were prepared and analyzed using GPC with a UV detector.

The chromatograph at 240 nm was analyzed for peak area.

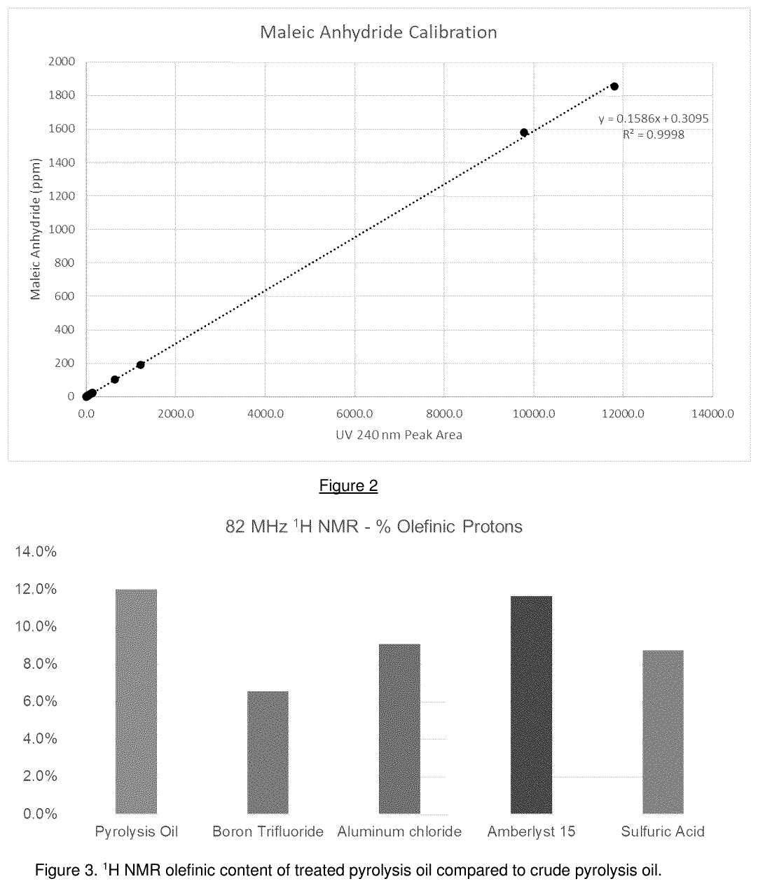

A plot of MA (ppm) versus peak area creates the calibration curve (FIG. 2). The equation of the line can be used to determine the concentration of MA in unknown solutions.

### ii) Preparation of Samples for Testing:

A solution of 6% wt maleic anhydride in toluene is prepared.

15 g of MA is added to 200 mL toluene and heated to 55° C. for 4 hours. The solution is then cooled, and the volume is brought to 250 mL.

The solution is let sit overnight and filtered the following day. This will remove some undissolved MA that has precipitated out.

Once made, the solution should be used within two weeks.

The concentration of the MA solution is checked using the equation from the GPC-UV calibration in the following (Equation 1.):

$$\frac{( \frac{0.1586 \times PA + 0.3095}{sample M . F .} )}{( \frac{Inj . Vol .}{5} )} = [ MA ] ( ppm ) \qquad Eq . 1$$

where PA is the peak area (15.8-16.6 min) at 240 nm (mV·s), sample M.F is the concentration of the sample

$$( \frac{sample ( g )}{sample ( g ) + THF ( g )} ) ,$$

and Inj. Vol is the sample injection volume (uL). The equation 0.1586x+0.3095 is the equation of the line from the MA calibration plot.
iii) Preparation of Sample and Diels-Alder (D-A) Reaction.

Weight out 15 g of sample (pyrolysis oil) and add to a 100 mL round bottom flask (RBF).

Add 20 mL of the 6% MA in toluene solution and a stir bar to the 100 mL RBF with the sample.

Mix the solution gently then remove 55-70 mg of the mixture and place into 20 mL vial. Record the exact amount of mixture added.

Add 9-10 g of THF to the 20 mL vial recording the exact amount of THE added.

The sample in THF in the 20 mL vial will be used for the initial determination of MA for the diene calculation. Notated as initial in calculations. Run GPC-UV.

The mixture in the 100 mL RBF is then heated to reflux for 3 hours.

Upon refluxing, 55-70 mg of the solution is then placed into a second 20 mL vial. Record the exact amount added.

Add 9-10 g of THF to this second 20 mL vial recording the exact amount of THF added. This sample is used for the final determination of MA for the diene calculations. Notated as final in calculations. Run GPC-UV.

Determining amount of reacted and unreacted MA.

The initial amount of MA is calculated using equation 1 and the initial sample.

The final amount of MA is calculated using equation 1 and the final sample.

The results are converted from ppm to % and the amount of MA consumed is calculated using Equation 2:

$$1 - \frac{Final MA \%}{Initial MA \%} = MA consumed \qquad Eq . 2$$

### Determining Diene % of the Sample.

To determine the diene content, it is needed to know the amount (g) of MA used during D-A reaction.

20 ml of 6% MA in toluene=1.8 g MA and 16.3 g toluene

Determine the percentage of C5H8 dienes and higher MW dienes.

Integration of the 240 nm peak area from 13.5-16 min and 16-18.5 min of the pure sample from GPC-UV gives the peak area percentages of the higher Mw dienes and the C5H8 dienes, respectively.

The Mw (g/mol) of the higher Mw dienes is determined using the Mw (g/mol) from the GPC and normalizing it using the Mw of the C5H8 peak and 68 g/mol.

Use Equation 3 to determine diene percentage.

$$[ \frac{( \frac{g MA \times \% {C}_{5} {H}_{8} \times MA Consumed}{Mw of MA ( \frac{g}{mol} )} ) \times Mw {C}_{5} {H}_{8} ( \frac{g}{mol} )}{grams of pyrolysis oil sample} ] + [ \frac{( \frac{g MA \times \% other dienes \times MA Consumed}{Mw of MA ( \frac{g}{mol} )} ) \times Mw other dienes ( \frac{g}{mol} )}{grams of pyrolysis oil sample} ] \qquad Eq . 3$$

The following pyrolysis plastic oils were used in the examples:

Pyrolysis Oil 1: it has a boiling point ranging from 50° C. to about 480° C. and a Diene value of about 2.7%. The chlorine content is in the range of about 115 ppm, the silicium content is expected in the range of about 15 ppm. The oxygen content is expected in the range of about 1 wt %. The nitrogen is expected in the range of about 1150 ppm wt.

Pyrolysis Oil 2: A light cut obtained by distillation of a pyrolysis oil (issued from a non-catalytic pyrolysis of mixed waste plastic) and having a boiling point ranging from about 50° C. to about 260° C. The water is expected to be below 100 ppm weight. The Diene value is expected to be in the range of about 4.8%.

Pyrolysis Oil 3: A light cut of non-catalytic pyrolysis plastic oil (obtained by staged condensation of the pyrolysis effluent) and having a boiling point ranging from <36° C. to about 260° C. This oil has been decanted to assure a water content below 100 ppm weight. The Diene value is expected to be in the range of about 7.8%.

Pyrolysis Oil 4: it has a boiling point ranging from 40° C. to about 401° C. and a Diene value of about 2.1%. The chlorine content is expected in the range of about 270 ppm, the silicium content is expected in the range of about 12 ppm. The oxygen content is expected in the range of about 0.3 wt %. The nitrogen is expected in the range of about 1250 ppm wt.

### Example 1. Production of a Liquid Resin or Oligomeric Product Using BF₃

The pyrolysis oil-based liquid resins were prepared through solution polymerization wherein the catalyst and monomers were continuously fed into the reaction vessel and boron trifluoride diethyl etherate is used as the catalyst with the BF₃ weight percentage on the total feed being 1.15+/−0.25%.

The flow rate of the monomer feed and the Friedel-Craft catalyst is controlled so that the catalyst feed is about 2 to 3 wt % on the combined mass flow of the monomer. The reaction temperature was maintained at 40° C. throughout the polymerization process and all steps carried out under a nitrogen blanket.

An initial amount of pyrolysis oil (−10 wt % of the total) was added as the reaction heel to a round bottom flask (RBF) purged with nitrogen. The heel was then warmed to 40° C. while stirring under nitrogen. The pyrolysis oil feed and Lewis acid catalyst (BF₃-OEt₂) were each measured out in individual flasks. If a comonomer was used (i.e. styrene) it was measured out and added to the flask with the pyrolysis oil feed. The pyrolysis oil and catalyst were fed into the reactor while maintain a temperature of 40° C. so that the catalyst was added at 2 to 3 wt % of the monomer and the two feeds finish adding at roughly the same time. Upon completion of the feed additions, the mixture was stirred at 40° C. for 45-60 minutes before cooling to room temperature. The polymerization was carried out under a nitrogen blanket.

The reaction mixture was then transferred to a separatory funnel and washed three times with ˜0.1M NH4OH, discarding the aqueous phase. The organic mixture was then washed twice more with water and the organic phase transferred to a separate three neck round bottom flask for distillation. During the washings, there is an insoluble tarry like residue that remains on the sides of the reaction flask and separatory funnel. In all cases of purification, the tarry residue was left behind unless otherwise noted.

The organic mixture was purged with nitrogen while being heated to 230° C. and the volatile organics were collected. Once the temperature reached 230° C. the distillation continued until there were no more volatiles being distilled at which point the nitrogen was replaced by steam. The volatiles collected prior to steam stripping are referred to as the distillate. Liquid resin or oligomeric product and steam condensate were collected until there was minimal liquid resin or oligomeric product being collected. The liquid resin or oligomeric product collected is referred to as oily polymer. The material remaining after steam stripping is a resin/wax product polymerized during purification. The distillate collected is the purified pyrolysis oil with the dienes reduced.

Table 1 shows the catalyst and comonomer amounts used for polymerization and the final diene concentration of the distillate following purification. The diene content has been reduced to less than 0.5% in all cases and in some instances, removed to undetectable levels, following polymerization.

TABLE 1
Initial and final diene concentration.

| | Poly 1 | Poly 2 | Poly 3 | Poly 4 | Poly 5 |
|---|---|---|---|---|---|
| Origin of the | Py Oil 1 | Py Oil 2 | Py Oil 2 | Py Oil 3 | Py Oil 3 |
| pyrolysis oil | | | | | |
| Initial Diene | 2.7 | 4.8 | 4.8 | 7.8 | 7.8 |
| Percent | | | | | |
| Styrene (wt %) | — | — | 1.4 | — | 9.0 |
| BF3, wt % (on | 0.92 | 1.16 | 1.24 | 1.40 | 1.28 |
| total feed) | | | | | |
| Final Diene | 0.4 | 0.0 | 0.3 | 0.2 | 0.0 |
| Percent | | | | | |

### Example 2. Production of an Oligomeric Product Using Aluminum Chloride (AlCl₃) as Catalyst

Hexanes (−10 wt % of the total) was added to a RBF purged with nitrogen. The AlCl₃ catalyst, approximately 1.25 wt % of the total feed, was then added to the hexanes and warmed to 35° C. while stirring under nitrogen. The pyrolysis oil feed was measured out in an individual flask and fed at a rate of 2 mL/min. Upon completion of the feed addition, the mixture was stirred at 35° C. for 50 minutes before cooling to room temperature. The polymerization was carried out under a nitrogen blanket.

The reaction mixture was then transferred to a separatory funnel and washed three times 25% aqueous isopropanol (IPA), discarding the aqueous phase. The organic mixture was then washed twice more with water and the organic phase transferred to a separate three neck round bottom flask for distillation. The organic mixture was then distilled, and steam stripped as in example 1.

### Example 3. Production of an Oligomeric Product Using Amberlyst 15 as Catalyst

Pyrolysis oil was added to a RBF and stirred under nitrogen while warming to 40° C. Amberlyst 15, approximately 1 wt % of the total feed, was added as the catalyst in two doses. The initial amount of Amberlyst 15 catalyst (⅔ of the total) was added once the oil reached 40° C. and then stirred at 40° C. for 45 minutes under nitrogen. The remaining ⅓ of catalyst was then added and the reaction stirred for an additional 75 minutes. The mixture was then cooled to room temperature and the catalyst filtered. The organic mixture was then distilled, and steam stripped as in example 1.

### Example 4. Production of an Oligomeric Product Using Sulfuric Acid (H₂SO₄)

Pyrolysis oil was added to an RBF and stirred at room temperature. Sulfuric acid, approximately 2.5 wt % on total feed, was then added to the stirring pyrolysis oil and there was an immediate exotherm. The reaction was stirred until the temperature dropped back to room temperature. The acid was neutralized, and the oil washed with dilute sodium carbonate (2×) followed by water (2×). The oil was then distilled, and steam stripped as in example 1.

The next table summarizes the various amounts of Lewis Acid Catalyst used in the examples 2 to 4.

TABLE 2
Summary of the Lewis Acid catalyst quantities used.

| | AlCl₃ | Amberlyst 15 | Sulfuric Acid |
|---|---|---|---|
| Hexanes (g) | 8.5 | — | — |
| Catalyst (g) | 0.95 | 0.84 | 1.25 |
| Py Oil 3 (g) | 75.6 | 86.2 | 63.8 |

For the examples 2 to 4, the impact of the purification process via cationic polymerization was measured via 82 MHz 1H NMR and FTIR as presented respectively on FIGS. 2 and 3. The comparison of the fraction attributed to olefinic protons in the pyrolysis oil with the same fraction after cationic polymerization shows a decrease of the fraction of olefinic protons with all acid catalyst used. A similar conclusion arises from the FIG. 3 where the conjugated olefin peak area decreases with the cationic polymerization. The examples 2 to 4 therefore demonstrate that the cationic polymerization allows to reduce the diene content with the catalyst AlCl₃, Amberlyst 15 and sulfuric acid.

### Example 5. Characterization of the Oligomeric Product

The resin or wax products derived from pyrolysis oil are a result of the cationic polymerization used on the pyrolysis oil. When the resin or wax is generated and isolated, then the resulting liquid hydrocarbon is a purified pyrolysis oil. The liquid oligomeric products are of various kind in those examples. Namely the wax+ product is constituted of the tarry residue that remains after the aqueous washes was dissolved in acetone/THF and added to the organic phase for distillation.

The isolated residue that remains following distillation and steam stripping of the post-polymerized pyrolysis oils are the resin or wax products dependent on the pyrolysis oil feed. The reaction conditions and product properties are shown in Table 3.

TABLE 3
Reaction Conditions and Product Properties of Polymerized Oils.

| | Wax+ | Wax | Resin | Resin-Sty |
|---|---|---|---|---|
| Pyrolysis Oil Lot | 1 | 1 | 2 | 2 |
| Initial Diene Percent | 2.7 | 2.7 | 4.8 | 4.8 |
| BF3, wt % (on total feed) | 0.92 | 0.92 | 1.16 | 1.24 |
| Pyrolysis Oil: Total (g) | 562.4 | 561.6 | 500.0 | 511.0 |
| Pyrolysis Oil: Heel (g) | 60.5 | 50.3 | 51.3 | 50.7 |
| Styrene Added (g) | — | — | — | 7.1 |
| Water Strip Amount (g) | 460.2 | 297.3 | 29.8 | 78.7 |
| Yield (%) | 12.0 | 25.8 | 4.2 | 5.2 |
| Final Diene Percent | 0.4 | 0.7 | 0.0 | 0.3 |
| Viscosity S27 (cps) | @33° C.: ~30 | @26° C.: ~30 | @25° C.: ~2,000 | @25° C.: ~2,000 |
| Final Resin T_{c}, ° C. | 27.2 | 24.3 | N/A | N/A |
| Final Resin Tₘ, ° C. | 30.1 | 26.0 | N/A | N/A |
| Final Resin T_{g}, ° C. | N/A | N/A | −45.5 | −38.5 |
| Mn (g/mol) | 470 | 424 | 345 | 348 |
| Mw (g/mol) | 1127 | 477 | 471 | 466 |
| Mz (g/mol) | 49430 | 524 | 749 | 729 |
| Mz + 1 (g/mol) | 152682 | 567 | 1431 | 1448 |
| MWD | 2.4 | 1.13 | 1.37 | 1.34 |
| Aromatic | 1.3% | 1.4% | 7.8% | 13.3% |
| Olefinic | 1.2% | 1.3% | 3.0% | 2.5% |
| Aliphatic | 97.5% | 97.3% | 89.2% | 84.2% |

As shown in Table 4, the properties of the oligomeric products are dependent on the pyrolysis oil feed. The aromatic content in the wax+ and wax products from pyrolysis oil 1 are relatively low and these are highly saturated hydrocarbon products. The melting and crystallization temperature of the wax+ and wax products being around room temperature. The resin and resin-sty products from the polymerization of pyrolysis oil 2 contain higher amount of aromatic content.

TABLE 4
Comparison of Polymerized and Commercial Products.

| | | | | | | Plaxolene |
|---|---|---|---|---|---|---|
| | Wax+ | Wax | Resin | Resin-Sty | Wingtack 10 | TD346 |
| Viscosity S27 | 33 C.: ~30 | 26 C.: ~30 | 25 C.: ~2,000 | 25 C.: ~2,000 | 25 C.: ~30,000 cps | 25 C.: ~5,000 |
| (cps) | | | | | | |
| T_{c} (° C.) | 27.2 | 24.3 | N/A | N/A | N/A | N/A |
| Tₘ (° C.) | 30.1 | 26.0 | N/A | N/A | N/A | N/A |
| T_{g} (° C.) | N/A | N/A | −45.5 | −38.5 | −32.8 | −50.5 |
| Mn (g/mol) | 470 | 424 | 345 | 348 | 505 | 383 |
| MWD | 2.4 | 1.13 | 1.37 | 1.34 | 1.22 | 1.39 |
| Aromatic | 1.3% | 1.4% | 7.8% | 13.3% | 0.0% | 5.2% |
| Olefinic | 1.2% | 1.3% | 3.0% | 2.5% | 3.7% | 0.0% |
| Aliphatic | 97.5% | 97.3% | 89.2% | 84.2% | 96.3% | 94.8% |

### Example 6. Preparation of an Adhesive Composition

The resin and wax products from the polymerization of pyrolysis oils 2 and 1 respectively were evaluated against Wingtack 10 (WT10, our reference) as processing aids in a basic tape formulation made up of the following components:

| Component | Wt. % |
|---|---|
| SBC-SIS Triblock Copolymer (Vector 4113A) | 43 |
| Tackifying Resin (WT98) | 50 |
| Processing Aid (Various) | 7 |
| Antioxidant (BNX 1010) | 1 |

Adhesive compositions were mixed in an open-top sigma blade mixer at 177° C. (350° F.) under a nitrogen blanket. All mixing times were kept to less than 60 minutes to minimize degradation of the styrenic block copolymer (SBC). First, the SBC and BNX 1010 antioxidant (AO) were combined and masticated for 5-10 minutes until smooth. The resin was added incrementally over several minutes to ensure good mixing and to prevent separation; once all the resin was added the formulation was mixed for 20-30 minutes. Finally, the processing aid was added slowly while mixing and then mixed for another 10 minutes. The adhesive was transferred to a suitable container coated with a release liner to facilitate easy removal once cool.

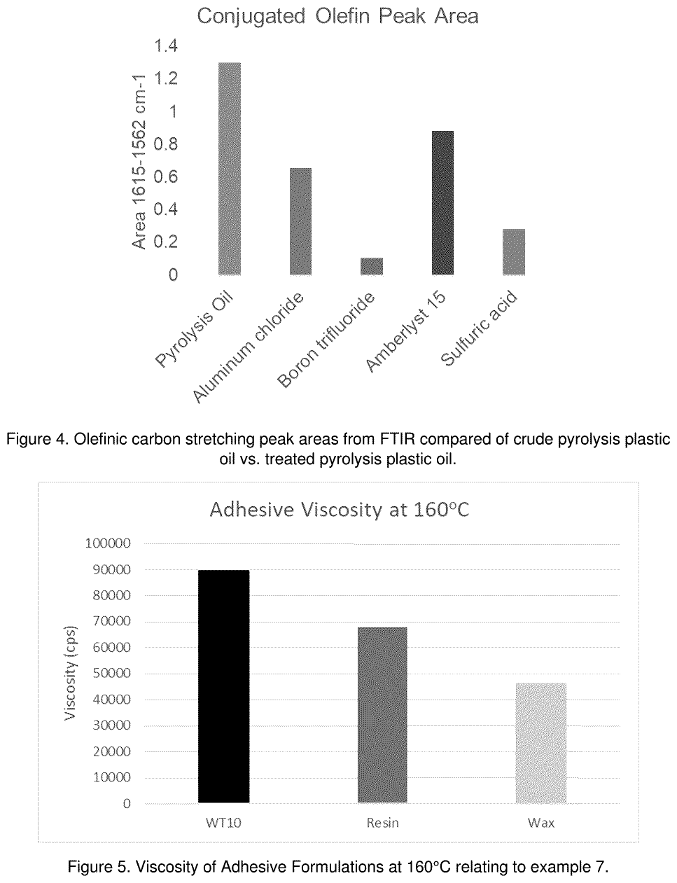

The viscosities of the adhesive formulations were measured at 160° C. and can be seen in FIG. 4. The viscosity of the formulations with the resin and wax are 24 and 48 percent less than the formulation using Wingtack10, respectively. The decrease in molten viscosity should allow for the reduction of the temperature needed for coating which translates to energy savings.

Wingtack 10 is a petroleum derived resin and was chosen as the control and is known to provide superior adhesive performance compared to process oil in low-Tg block polymer PSAs, without creating the migration or shelf-life problems that process oils frequently cause. Therefore, performance of the pyrolysis-oil based resin superior to or equal to that of the control is indicative of improved performance relative to common processing oils.

The adhesives were coated onto 2 mil (50 micron) PET at an approximate thickness of 0.9 mils (22 microns) dry. The adhesive coated PET was laminated to release paper and allowed to condition at 50% relative humidity and 23° C. (73° F.) a minimum of 24 hours before initial testing. For aged sample testing, the adhesive coated PET laminated to release paper was placed in the oven at 70° C. for 7 days before testing. One inch and one-half inch strips were cut and tested according to methods detailed by the Pressure Sensitive Tape Council (PSTC).

180° peel adhesion testing was conducted using one-inch wide (2.54 cm) test specimen and stainless steel (SS), corrugated cardboard (CC), and high-density polyethylene (HDPE) test substrates. All adhesion test results are reported in pounds per square inch (psi). Loop tack and quick stick testing were conducted using the same three test substrates and reported in psi. Room temperature shear testing was conducted using specimens that were 0.5″ wide with an overlap of 0.5″ (total ¼ in² contact area) and were tested using 0.5 kg weights and SS panels as well as unbleached kraft paper held to the test panels with double-sided tape. Heat resistance was determined by measuring the shear adhesion failure temperature (SAFT) and reported in ° C. Test specimens for SAFT determination were one inch wide and had a contact area of 1 in² with a weight of 1 kg. All testing was conducted in triplicate and the averages reported.

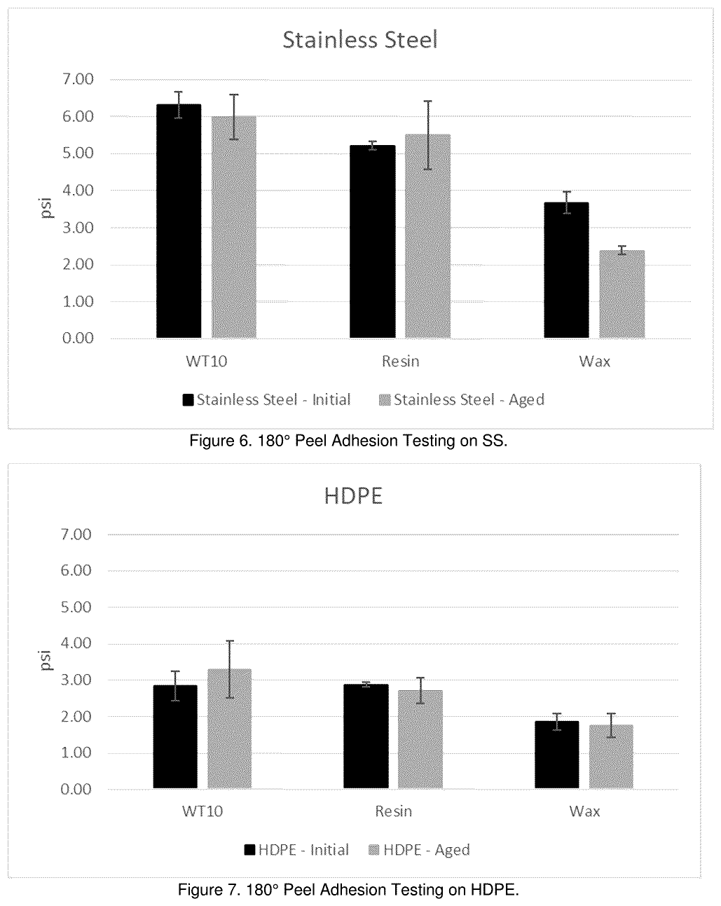

FIG. 5, FIG. 6, and FIG. 7 show the results of the 180° peel adhesion testing on SS, CC, and HDPE for the initial and aged samples. The adhesion values for the resin-based adhesive is comparable to the control-based adhesive within the experimental error involved in the testing. The adhesion values for the wax-based adhesive is comparable, within experimental error, to the control on CC.

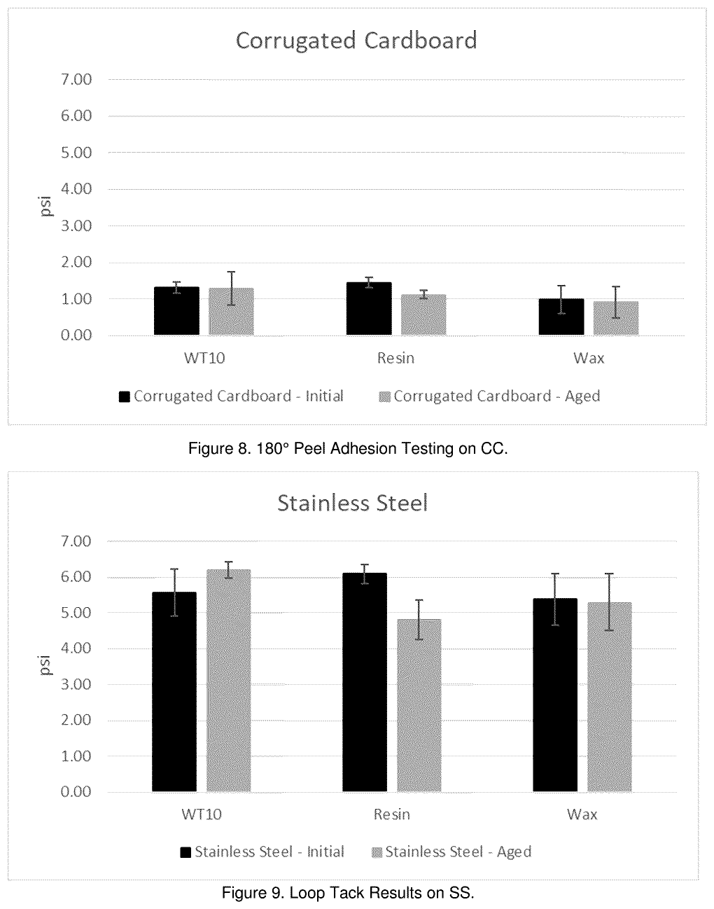

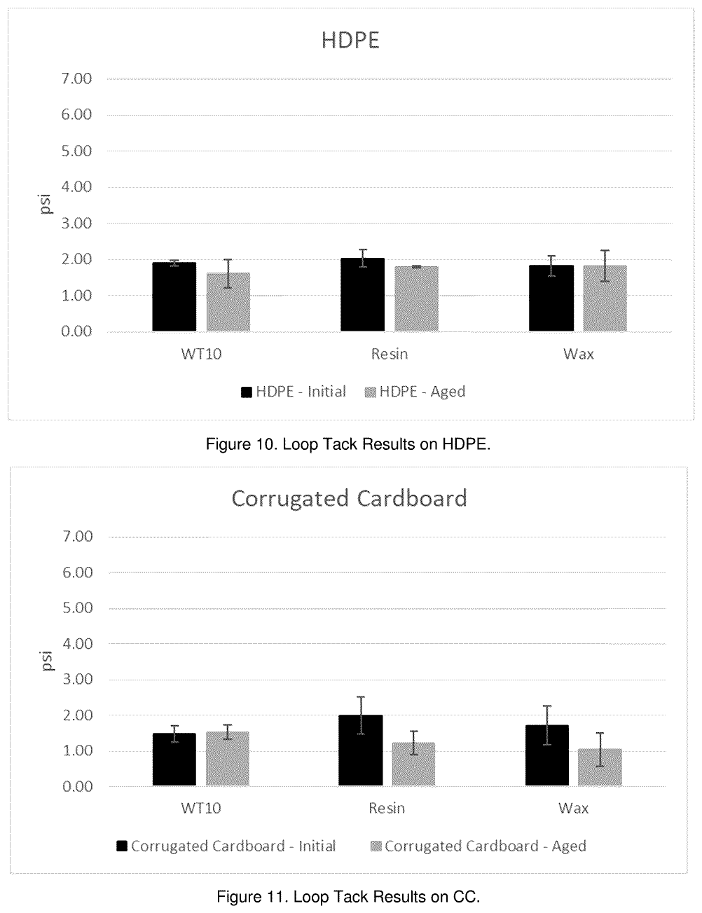

As shown in FIG. 8, FIG. 9, and FIG. 10 the loop tack data comparing the adhesives with the resin and wax products from pyrolysis oil polymerization to the adhesive with Wingtack 10 exhibit similar performance on SS, HDPE, and CC.

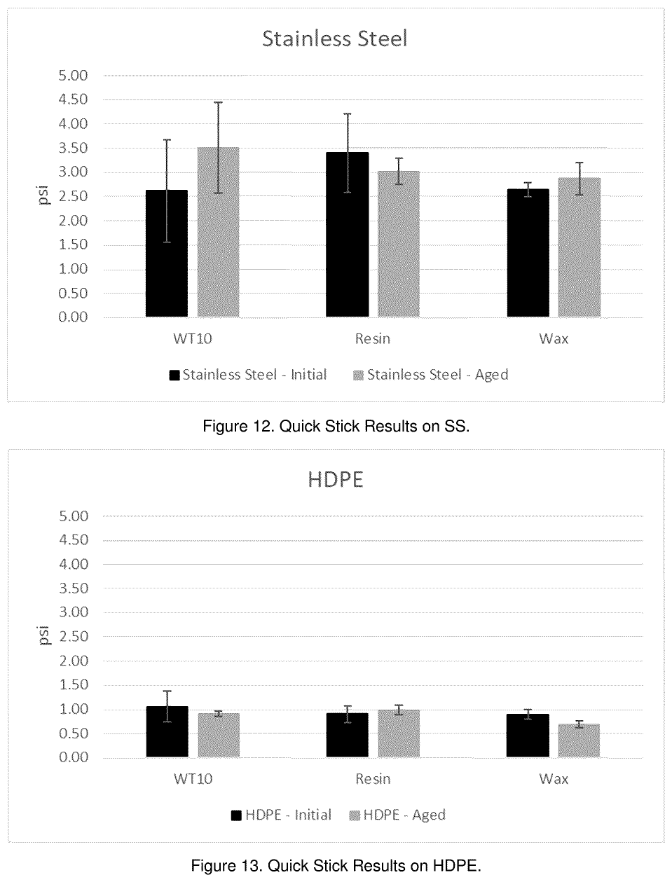

FIG. 11, FIG. 12, and FIG. 13 show the quick stick results and similar performance of the resin and wax-based adhesives to the WT10 based adhesive on SS, HDPE, and CC. The adhesives formulated with the resin and WT10 exhibit stick slip on SS and HDPE during the quick stick test while the adhesive formulated with the wax does not have any stick slip on any of the tested substrates. The results from the loop tack and quick stick tests indicate that the adhesives formulated using the resin and wax from the pyrolysis oil polymerizations have similar tack to the adhesive formulated using WT10.

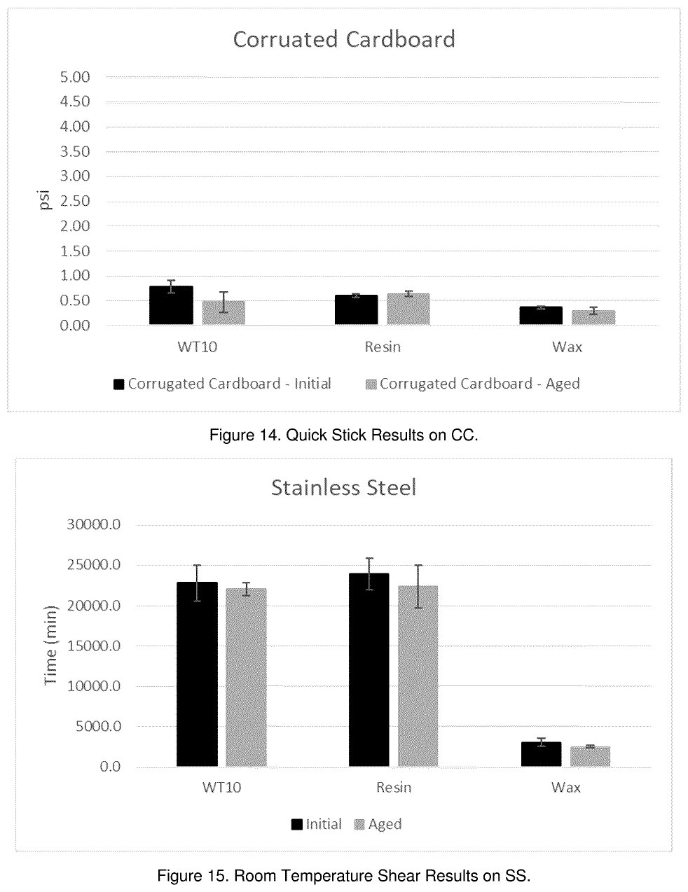

The results of the room temperature shear testing on SS and kraft paper are shown in FIG. 14 and FIG. 15. All samples that remained hanging after 25,000 minutes were removed from the test equipment and the result was reported as 25,000 minutes. The performance of the resin-based adhesive is comparable to the WT10 based adhesive on both SS and kraft paper.

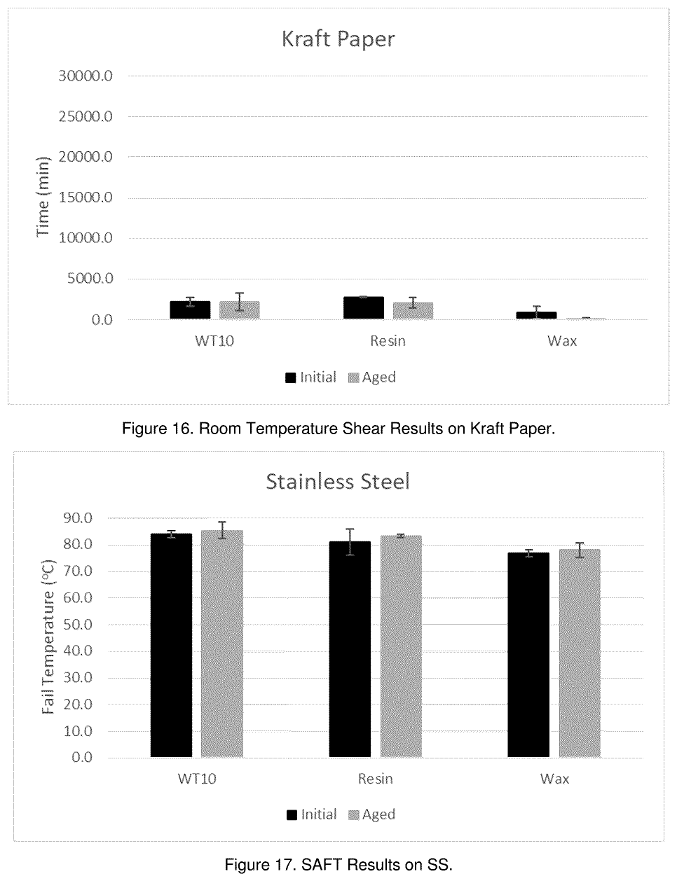

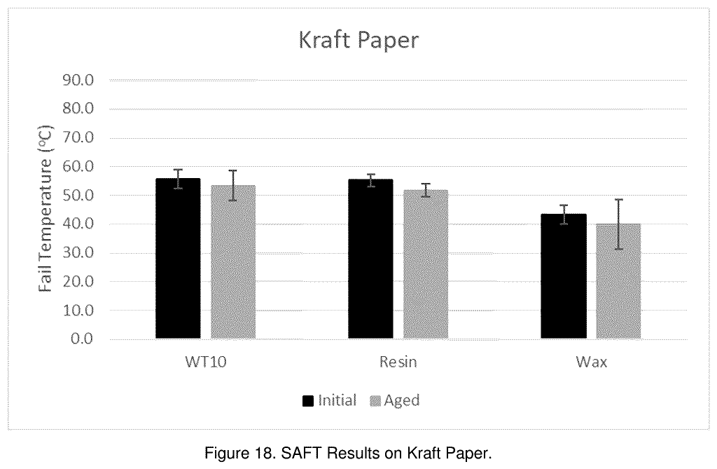

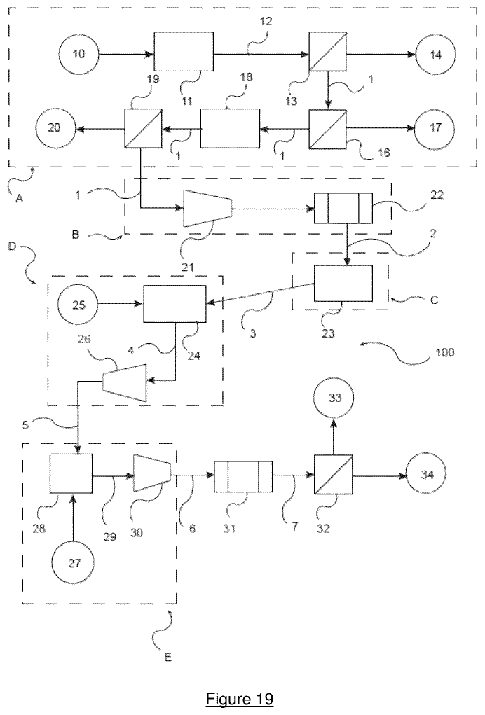

The heat resistance of the adhesives formulated using the resin, wax, and WT10 as measured by SAFT are shown in FIG. 16 and FIG. 17. The heat resistance of all three adhesive formulations is the same with experimental error on SS, and the resin material is similar on Kraft paper.

1-34. (canceled)

35. An adhesive composition comprising an elastomer and one or more tackifying resins is characterised in that one of the one or more tackifying resins comprises a liquid resin prepared from a liquified waste polymer, being preferably a pyrolysis oil via polymerization.

36. The adhesive composition according to claim 35 is characterized in that the elastomer is selected from the group consisting of styrene-isoprene block copolymers, polyacrylate resins, poly ethylene vinyl acetate (EVA) resins, polystyrene butadiene resins, random styrenebutadiene (SBR) copolymers, styrene-butadiene block copolymers, styrene-isoprene-butadiene-styrene (SIBS) copolymers, styrene-ethylene-propylene-styrene (SEPS) copolymers, styrene-ethylene-butylene-styrene (SEBS) block copolymers, amorphous poly-olefin (APO) resins, and mixtures thereof

37. The adhesive composition according to claim 35 is characterized in that the content of said elastomer ranges from 1 to 90 wt. %, and/or the content of tackifying resin derived from liquid waste polymer ranges from 1 to 75 wt. %.

38. The adhesive composition according to claim 35 is characterized in that the liquid resin shows an olefinic content of at most 20.0 wt. % as determined by 1H NMR; preferably ranging from 0.1 to 10.0 wt. %.

39. The adhesive composition according to claim 35 is characterized in that the liquid resin shows an aliphatic content ranging from 15 to 100 wt. % as determined by 1H NMR; preferably ranging from 50.0 to 99.0 wt. %.

40. The adhesive composition according to claim 35 is characterized in that the liquid resin presents an aromatic content below 50.0 wt. % as determined by 1H NMR; preferably ranging from 0.1 to 50.0 wt. %.

41. The adhesive composition according to claim 35 is characterized in that the liquid resin presents a viscosity at 30° C. ranging from 10 to 50 000 cps measured using a Brookfield viscometer; preferably ranging from 10 to 10 000 cps.

42. The adhesive composition according to claim 35 is characterized in that the liquid resin presents a number average molecular weight (Mn) ranging from 100 to 10 000 g/mol measured using gel permeation chromatography and a polystyrene calibration; preferably from 100 to 700 g/mol.

43. The adhesive composition according to claim 35 is characterized in that the liquid resin comprises a comonomer selected from the group consisting of styrene, alpha-methyl styrene, 2-methyl styrene, 3-methyl styrene, 4-methyl styrene, 4-t-butyl styrene, 4-cyclohexyl styrene, 4-dodecyl styrene, 2,4-diisopropyl styrene, 2.4.6-trimethyl styrene, 2-ethyl-4-benzylstyrene, 4-(phenylbutyl) styrene, 1-vinyl naphthalene, 2-vinyl naphthalene, vinyl anthracene, 4-methoxy styrene, monochlorostyrene, dichlorostyrene, divinyl benzene, Indene, methyl-Indene, and mixtures thereof; with preference liquid resin comprises a comonomer being or comprising styrene.

44. The adhesive composition according to claim 35 is characterized in that it is a wax and/or it shows a crystallisation temperature (Tc) below 35° C.; preferably ranging from 20 to 30° C.

45. The adhesive composition according to claim 35 is characterized in that it is a resin and/or it shows a glass transition temperature (Tg) below −20° C. as determined by Differential Scanning calorimetry; preferably ranging from −50° C. to −30° C.

46. An article comprising a substrate and the adhesive composition according to claim 35, the adhesive composition being applied to at least one surface of the substrate, wherein the substrate is selected from the group consisting of a tape and a label.

47. A process for the preparation of a liquid resin from a liquified waste polymer, the liquid resin being an oligomeric product and being suitable to prepare an adhesive composition according to claim 35 or in any product according to claim 46, the process is characterized in the following steps:
a) providing a feedstream (1) containing liquified waste polymer, wherein said feedstream (1) contains at least 0.1 wt. % to at most 80 wt. % of dienes based on the total weight of said feedstream (1);
c) performing a polymerization reaction on said feedstream (1) under polymerization conditions to obtain a first product stream (3) comprising an oligomeric product (33) and a purified liquified waste polymer;
d) optionally, performing a neutralization reaction by contacting said first product stream (3) with a basic compound (25) to obtain a neutralized product stream (4) and removing said basic compound from neutralized product stream (4) to obtain a second product stream (5);
g) performing a separation to separate the oligomeric product (33) from the purified liquified waste polymer (34).

48. The process according to claim 47 is characterized in that the polymerization reaction in step c) is a cationic polymerization; and/or in that the process further comprises a step (b) of drying the feedstream (1) to obtain a dried feedstream (2) wherein step b) is performed before step c) of polymerization so that step c) of polymerization reaction is performed on the dried feedstream (2).

49. The process according to claim 47 is characterized in that the feedstream contains at least 75 wt. % of the liquefied waste polymer based on the total weight of the feed stream; and/or in that the polymerization reaction in step c) is a cationic polymerization performed in the presence of an acidic catalyst; with preference, the acidic catalyst is a Brönsted acid or a Lewis acid.

50. The process according to claim 47 is characterized in that the polymerization conditions of step (c) comprises a contact time ranging from 5 min to 5 hours; and/or a temperature ranging from 5 to 100° C. at atmospheric pressure.

51. The process according to claim 47 is characterized in that it further comprises a step e) of washing the first product stream (3) or the second product stream (5) with a solvent (27) to obtain a washed stream (6); with preference, the washing is performed at a temperature ranging from 5° C. to 95° C.

52. The process according to claim 47 is characterized in that it further comprises a step f) of filtering the stream obtained in the previous step to obtain a filtered stream (7) wherein the filtering is performed to remove solids from the first product stream (3) or from the second product stream (5) or from the washed stream (6), and/or to coalesce remaining traces of solvent if any; with preference, the filtering step is followed by a dewatering step.

53. The process according to claim 47 is characterized in that, after the separation step g), the purified liquified waste polymer is recovered and blended in the fuel pool; with preference, the purified liquified waste polymer is separated in a naphtha cut having a boiling range of less than 150° C. and a diesel cut having a boiling range between 15° and 350° C., wherein said naphtha cut is incorporated in a naphtha pool, said diesel cut is incorporated in a diesel pool.

54. The process according to claim 47 is characterized in that the liquified waste polymer in the feedstream (1) has a final boiling point of at most 700° C. and/or feedstream (1) contains from 0.1 to 25.0 wt. % of dienes based on the total weight of said feedstream (1).
